(19) United States
(12) Patent Application Publication
(10) Pub. No.: US 2024/0252449 A1
(43) Pub. Date: Aug. 1, 2024

(54) STABILISED COMPOSITIONS

(71) Applicant: Rumin8 Pty Ltd, Mount Pleasant (AU)

(72) Inventors: Cameron Jay SCADDING, Landsdale (AU); Matthew CALLAHAN, Haverford, PA (US)

(21) Appl. No.: 18/431,461

(22) Filed: Feb. 2, 2024

Related U.S. Application Data
(63) Continuation of application No. PCT/AU2022/050836, filed on Aug. 3, 2022.

(30) Foreign Application Priority Data
Aug. 3, 2021 (AU) 2021902391

(51) Int. Cl. A61K 31/02; A61K 9/00; A61K 9/107; A61K 9/127; A61P 1/14

(52) U.S. Cl. CPC A61K 31/02; A61K 9/0056; A61K 9/107; A61K 9/1075; A61K 9/127; A61P 1/14

(57) ABSTRACT

Compositions for reducing or inhibiting methanogenesis in ruminants comprising, at least one halocarbon compound and one or more stabilising agents.

### RELATED APPLICATIONS

This application is a continuation of International Application No. PCT/AU2022/050836, filed on Aug. 3, 2022, which claims priority to Australian Application No. 2021902391, filed Aug. 3, 2021, each of which is hereby incorporated by reference in their entirety.

### TECHNICAL FIELD

The technical field of the invention relates to stabilised compositions for reducing and/or inhibiting methanogenesis in the digestive tract of ruminants, including processes for their production, and methods for their administration to ruminants.

### BACKGROUND ART

The following discussion of the background art is intended to facilitate an understanding of the present invention only. The discussion is not an acknowledgement or admission that any of the material referred to is or was part of the common general knowledge as at the priority date of the application.

Cattle-rearing and ruminant livestock agriculture in general produces more global warming greenhouse gases, as measured in carbon dioxide (CO₂) equivalents, than transportation, according to a recent UN assessment. Ruminants are estimated to account for 10% of total greenhouse gas emissions in Australia. Ruminants produce methane (CH₄) as a by-product of digestion via anaerobic microbial feed fermentation in the rumen and, to a lesser extent, the large intestine. This process is referred to as methanogenesis.

Methane absorbs solar infrared radiation efficiently, and has a global warming potential 25 times that of CO₂. The ruminal microbial population is made up of bacteria, protozoa, fungi, and bacteriophages, all of which work together to digest ingested organic matter and produce CO₂, H₂, volatile fatty acids, and formates. These end-products are used by methanogenic archaea in the rumen, which produces CH₄. Although the generation of CH₄ lowers the partial pressure of H₂, this has the potential to cause problems as it also limits the amount of energy and carbon available for the synthesis of volatile fatty acids, which are critical for ruminant nutrition and could otherwise restrict rumen fermentation. The majority of CH₄ generated by ruminants is exhaled or discharged via the mouth, resulting in a waste of up to 12% of gross caloric intake in the ruminant diet.

Enteric methanogenesis mitigation techniques are crucial to the ongoing economic and environmental viability of ruminant livestock agriculture industries, and methods of lowering total gas production and/or methane generation in ruminant animals pose a serious challenge.

Among several chemical and plant-derived inhibitors and prospective methanogenesis reducing diets tested, the natural antimethanogenic red seaweed macroalga Asparagopsis taxiformis has recently shown promise, with dose-dependent, methane inhibiting effects that have minimal impacts on other rumen fermentation parameters. However, as with any naturally derived plant-based feed supplement, challenges are posed by a lack of sustainable commercial scale production, as well as inconsistencies in biological activity from sample to sample arising from variations in phenotype and/or chemotype from samples obtained from differing locations, and due to variable environmental conditions. Such variability introduces significant uncertainties surrounding safe and effective dosage regimes in these plant-based solutions to limiting methanogenesis in ruminants.

Inhibition of methanogenesis in ruminants via supplementation of their feed with Asparagopsis taxiformis is largely attributed to the compound bromoform, which is present in the essential oil of the seaweed at concentrations as high as 80% (w/w), along with numerous other bromine- chlorine- and iodine-containing organic compounds including bromomethane, bromochloromethane, and other volatile halogenated compounds. A. taxiformis contains between 0.19 and 4.39 mg/g (dry weight) bromoform.

Direct administration of synthetic bromoform as a feed supplement to inhibit methanogenesis in ruminants therefore represents an attractive prospect for addressing the challenges discussed above in relation to the use of seaweed products as a feed supplement. However, pure or synthetic bromoform poses a number of alternative problems, particularly due to its volatility and associated environmental toxicity. Furthermore, bromine delivered to the atmosphere by volatile compounds such as bromoform is believed to play a significant role in participating in ozone-depleting chemistry, further exacerbating the global warming problems we now face.

It is against this background that the present invention has been developed.

### SUMMARY OF THE INVENTION

In one embodiment, the disclosure herein provides a composition for reducing or inhibiting methanogenesis in ruminants comprising, a halocarbon compound and one or more stabilising agents.

In a preferred embodiment, the composition does not comprise red marine macroalgae or a halocarbon producing recombinant yeast, or the halocarbon compound is not an extract of red marine macroalgae, or an extract of a halocarbon producing recombinant yeast, or chloroform, or carbon tetrachloride, or methylene chloride, or hexachloroethane, or bromochloromethane, or bromochloroethane, or bromochloropropane, or halothane, or 2-bromoethane sulphonate, or 2-bromo-2-chloroethane sulphonate.

In one embodiment, the disclosure herein provides a composition for reducing or inhibiting methanogenesis in ruminants comprising a halocarbon compound, wherein the halocarbon compound is stabilised.

In one embodiment, the disclosure herein provides a stabilised composition for reducing or inhibiting methanogenesis in ruminants comprising, a halocarbon compound selected from the group consisting of haloalkanes, haloalkenes, haloaromatics, halogenated carboxylic acids and their corresponding esters, haloalkyl alcohols, haloalkyl ethers, haloalkyl amines and salts thereof, haloalkyl thioethers and haloalkyl amides, including mixtures of any of the aforesaid halocarbon compounds, and/or metabolites of any of the aforesaid halocarbon compounds.

In a preferred embodiment, the disclosure herein provides a stabilised composition for reducing or inhibiting methanogenesis in ruminants comprising, bromoform.

In a preferred embodiment, the disclosure herein provides a stabilised composition for reducing or inhibiting methanogenesis in ruminants comprising, stabilised bromoform.

In one embodiment, the disclosure herein provides a stabilised composition for reducing or inhibiting methanogenesis in ruminants comprising a halocarbon compound, wherein the halocarbon compound is stabilised in terms of having reduced volatility in the composition, compared to the volatility observed in the isolated halocarbon.

In a preferred embodiment, the disclosure herein provides a stabilised composition for reducing or inhibiting methanogenesis in ruminants comprising, stabilised bromoform wherein the bromoform is stabilised in terms of having reduced volatility in the composition, compared to the volatility observed in isolated bromoform.

In some embodiments, the stabilisation of the halocarbon compound in the compositions of the invention, is stabilisation in terms of having reduced volatility in the composition, compared to the volatility observed in the isolated halocarbon, preferably wherein the reduced volatility is measured via comparing a measurement of the vapour pressure of the composition to a measurement of the vapour pressure of the isolated halocarbon, wherein both measurements are taken at the same temperature.

In preferred embodiments, the stabilisation of the bromoform in the compositions of the invention, is stabilisation in terms of having reduced volatility in the composition, compared to the volatility observed in isolated bromoform, preferably wherein the reduced volatility is measured via comparing a measurement of the vapour pressure of the composition to a measurement of the vapour pressure of the isolated bromoform, wherein both measurements are taken at the same temperature.

In some embodiments, the stabilisation of the halocarbon compound in the compositions of the invention, is stabilisation in terms of having reduced volatility in the composition, compared to the volatility observed in the isolated halocarbon, wherein the reduced volatility is measured via comparing the % loss of the halocarbon compound from the composition over a period of time, with the % loss of isolated halocarbon from a sample of the isolated halocarbon over the same period of time, and wherein both the composition and the isolated halocarbon are stored under identical conditions; preferably wherein the % loss of the halocarbon compound from the composition over a period of time is measured via one or more methods selected from the group consisting of GC-MS, or LC-MS, GC-FID or ICP-AES; preferably wherein the % loss of the sample of the isolated halocarbon over the same period of time is measured via gravimetric analysis.

In preferred embodiments, the stabilisation of the bromoform in the compositions of the invention, is stabilisation in terms of having reduced volatility in the composition, compared to the volatility observed in isolated bromoform, wherein the reduced volatility is measured via comparing the % loss of the bromoform from the composition over a period of time, with the % loss of bromoform from a sample of isolated bromoform over the same period of time, and wherein both the composition and the isolated bromoform are stored under identical conditions; preferably wherein the % loss of bromoform from the composition over a period of time is measured via one or more methods selected from the group consisting of GC-MS, or LC-MS, GC-FID or ICP-AES; preferably wherein the % loss of the sample of isolated bromoform over the same period of time is measured via gravimetric analysis.

In some embodiments, the stabilisation of the halocarbon compound in the compositions of the invention, is stabilisation in terms of having reduced loss of the halocarbon compound in the composition, compared to the loss observed in the isolated halocarbon, wherein the reduced loss is measured via comparing the % loss of the halocarbon compound from the composition over a period of time, with the % loss of isolated halocarbon from a sample of the isolated halocarbon over the same period of time, and wherein both the composition and the isolated halocarbon are stored under identical conditions; preferably wherein the % loss of the halocarbon compound from the composition over a period of time is measured via one or more methods selected from the group consisting of GC-MS, or LC-MS, GC-FID or ICP-AES: optionally wherein the % loss of the sample of the isolated halocarbon over the same period of time is measured via one or more methods selected from the group consisting of GC-MS, or LC-MS, GC-FID or ICP-AES; preferably wherein the % loss of the sample of the isolated halocarbon over the same period of time is measured via gravimetric analysis.

In preferred embodiments, the stabilisation of the bromoform in the compositions of the invention, is stabilisation in terms of having reduced loss of bromoform in the composition, compared to the loss observed in isolated bromoform, wherein the reduced loss is measured via comparing the % loss of bromoform from the composition over a period of time, with the % loss of isolated bromoform from a sample of isolated bromoform over the same period of time, and wherein both the composition and the isolated bromoform are stored under identical conditions; preferably wherein the % loss of the bromoform from the composition over a period of time is measured via one or more methods selected from the group consisting of GC-MS, or LC-MS, GC-FID or ICP-AES; optionally wherein the % loss of the sample of the isolated bromoform over the same period of time is measured via one or more methods selected from the group consisting of GC-MS, or LC-MS, GC-FID or ICP-AES; preferably wherein the % loss of the sample of the isolated bromoform over the same period of time is measured via gravimetric analysis.

In some embodiments, the halocarbon compound in the compositions of the invention is X % more stable over a period of time t, than the corresponding isolated halocarbon compound, wherein X % falls within the range of about 1% to about 5000%, and t is an integer representing a number of days.

In preferred embodiments, the bromoform in the compositions of the invention is X % more stable over a period of time t, than isolated bromoform, wherein X % falls within the range of about 1% to about 5000%, and t is an integer representing a number of days.

In some embodiments, X % (more stable) is selected from the group consisting of 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295, 296, 297, 298, 299, 300, 301, 302, 303, 304, 305, 306, 307, 308, 309, 310, 311, 312, 313, 314, 315, 316, 317, 318, 319, 320, 321, 322, 323, 324, 325, 326, 327, 328, 329, 330, 331, 332, 334, 335, 336, 337, 338, 339, 340, 341, 342, 343, 344, 345, 346, 347, 348, 349, 350, 351, 352, 353, 354, 355, 356, 357, 358, 359, 360, 361, 362, 363, 364, 365, 366, 367, 368, 369, 370, 371, 372, 373, 374, 375, 376, 377, 378, 379, 380, 381, 382, 383, 384, 385, 386, 387, 388, 389, 390, 391, 392, 393, 394, 395, 396, 397, 398, 399, 400, 401, 402, 403, 404, 405, 406, 407, 408, 409, 410, 411, 412, 413, 414, 415, 416, 417, 418, 419, 420, 421, 422, 423, 424, 425, 426, 427, 428, 429, 430, 431, 432, 433, 434, 435, 436, 437, 438, 439, 440, 441, 442, 443, 444, 445, 446, 447, 448, 449, 450, 451, 452, 453, 454, 455, 456, 457, 458, 459, 460, 461, 462, 463, 464, 465, 466, 467, 468, 469, 470, 471, 472, 473, 474, 475, 476, 477, 478, 479, 480, 481, 482, 483, 484, 485, 486, 487, 488, 489, 490, 491, 492, 493, 494, 495, 496, 497, 498, 499, 500, 501, 502, 503, 504, 505, 506, 507, 508, 509, 510, 511, 512, 513, 514, 515, 516, 517, 518, 519, 520, 521, 522, 523, 524, 525, 526, 527, 528, 529, 530, 531, 532, 533, 534, 535, 536, 537, 538, 539, 540, 541, 542, 543, 544, 545, 546, 547, 548, 549, 550, 551, 552, 553, 554, 555, 556, 557, 558, 559, 560, 561, 562, 563, 564, 565, 566, 567, 568, 569, 570, 571, 572, 573, 574, 575, 576, 577, 578, 579, 580, 581, 582, 583, 584, 585, 586, 587, 588, 589, 590, 591, 592, 593, 594, 595, 596, 597, 598, 599, 600, 601, 602, 603, 604, 605, 606, 607, 608, 609, 610, 611, 612, 613, 614, 615, 616, 617, 618, 619, 620, 621, 622, 623, 624, 625, 626, 627, 628, 629, 630, 631, 632, 633, 634, 635, 636, 637, 638, 639, 640, 641, 642, 643, 644, 645, 646, 647, 648, 649, 650, 651, 652, 653, 654, 655, 656, 657, 658, 659, 660, 661, 662, 663, 664, 665, 666, 667, 668, 669, 670, 671, 672, 673, 674, 675, 676, 677, 678, 679, 680, 681, 682, 683, 684, 685, 686, 687, 688, 689, 690, 691, 692, 693, 694, 695, 696, 697, 698, 699, 700, 701, 702, 703, 704, 705, 706, 707, 708, 709, 710, 711, 712, 713, 714, 715, 716, 717, 718, 719, 720, 721, 722, 723, 724, 725, 726, 727, 728, 729, 730, 731, 732, 733, 734, 735, 736, 737, 738, 739, 740, 741, 742, 743, 744, 745, 746, 747, 748, 749, 750, 751, 752, 753, 754, 755, 756, 757, 758, 759, 760, 761, 762, 763, 764, 765, 766, 767, 768, 769, 770, 771, 772, 773, 774, 775, 776, 777, 778, 779, 780, 781, 782, 783, 784, 785, 786, 787, 788, 789, 790, 791, 792, 793, 794, 795, 796, 797, 798, 799, 800, 801, 802, 803, 804, 805, 806, 807, 808, 809, 810, 811, 812, 813, 814, 815, 816, 817, 818, 819, 820, 821, 822, 823, 824, 825, 826, 827, 828, 829, 830, 831, 832, 833, 834, 835, 836, 837, 838, 839, 840, 841, 842, 843, 844, 845, 846, 847, 848, 849, 850, 851, 852, 853, 854, 855, 856, 857, 858, 859, 860, 861, 862, 863, 864, 865, 866, 867, 868, 869, 870, 871, 872, 873, 874, 875, 876, 877, 878, 879, 880, 881, 882, 883, 884, 885, 886, 887, 888, 889, 890, 891, 892, 893, 894, 895, 896, 897, 898, 899, 900, 901, 902, 903, 904, 905, 906, 907, 908, 909, 910, 911, 912, 913, 914, 915, 916, 917, 918, 919, 920, 921, 922, 923, 924, 925, 926, 927, 928, 929, 930, 931, 932, 933, 934, 935, 936, 937, 938, 939, 940, 941, 942, 943, 944, 945, 946, 947, 948, 949, 950, 951, 952, 953, 954, 955, 956, 957, 958, 959, 960, 961, 962, 963, 964, 965, 966, 967, 968, 969, 970, 971, 972, 973, 974, 975, 976, 977, 978, 979, 980, 981, 982, 983, 984, 985, 986, 987, 988, 989, 990, 991, 992, 993, 994, 995, 996, 997, 998, 999, 1000, 1001, 1002, 1003, 1004, 1005, 1006, 1007, 1008, 1009, 1010, 1011, 1012, 1013, 1014, 1015, 1016, 1017, 1018, 1019, 1020, 1021, 1022, 1023, 1024, 1025, 1026, 1027, 1028, 1029, 1030, 1031, 1032, 1033, 1034, 1035, 1036, 1037, 1038, 1039, 1040, 1041, 1042, 1043, 1044, 1045, 1046, 1047, 1048, 1049, 1050, 1051, 1052, 1053, 1054, 1055, 1056, 1057, 1058, 1059, 1060, 1061, 1062, 1063, 1064, 1065, 1066, 1067, 1068, 1069, 1070, 1071, 1072, 1073, 1074, 1075, 1076, 1077, 1078, 1079, 1080, 1081, 1082, 1083, 1084, 1085, 1086, 1087, 1088, 1089, 1090, 1091, 1092, 1093, 1094, 1095, 1096, 1097, 1098, 1099, 1100, 1101, 1102, 1103, 1104, 1105, 1106, 1107, 1108, 1109, 1110, 1111, 1112, 1113, 1114, 1115, 1116, 1117, 1118, 1119, 1120, 1121, 1122, 1123, 1124, 1125, 1126, 1127, 1128, 1129, 1130, 1131, 1132, 1133, 1134, 1135, 1136, 1137, 1138, 1139, 1140, 1141, 1142, 1143, 1144, 1145, 1146, 1147, 1148, 1149, 1150, 1151, 1152, 1153, 1154, 1155, 1156, 1157, 1158, 1159, 1160, 1161, 1162, 1163, 1164, 1165, 1166, 1167, 1168, 1169, 1170, 1171, 1172, 1173, 1174, 1175, 1176, 1177, 1178, 1179, 1180, 1181, 1182, 1183, 1184, 1185, 1186, 1187, 1188, 1189, 1190, 1191, 1192, 1193, 1194, 1195, 1196, 1197, 1198, 1199, 1200, 1201, 1202, 1203, 1204, 1205, 1206, 1207, 1208, 1209, 1210, 1211, 1212, 1213, 1214, 1215, 1216, 1217, 1218, 1219, 1220, 1221, 1222, 1223, 1224, 1225, 1226, 1227, 1228, 1229, 1230, 1231, 1232, 1233, 1234, 1235, 1236, 1237, 1238, 1239, 1240, 1241, 1242, 1243, 1244, 1245, 1246, 1247, 1248, 1249, 1250, 1251, 1252, 1253, 1254, 1255, 1256, 1257, 1258, 1259, 1260, 1261, 1262, 1263, 1264, 1265, 1266, 1267, 1268, 1269, 1270, 1271, 1272, 1273, 1274, 1275, 1276, 1277, 1278, 1279, 1280, 1281, 1282, 1283, 1284, 1285, 1286, 1287, 1288, 1289, 1290, 1291, 1292, 1293, 1294, 1295, 1296, 1297, 1298, 1299, 1300, 1301, 1302, 1303, 1304, 1305, 1306, 1307, 1308, 1309, 1310, 1311, 1312, 1313, 1314, 1315, 1316, 1317, 1318, 1319, 1320, 1321, 1322, 1323, 1324, 1325, 1326, 1327, 1328, 1329, 1330, 1331, 1332, 1333, 1334, 1335, 1336, 1337, 1338, 1339, 1340, 1341, 1342, 1343, 1344, 1345, 1346, 1347, 1348, 1349, 1350, 1351, 1352, 1353, 1354, 1355, 1356, 1357, 1358, 1359, 1360, 1361, 1362, 1363, 1364, 1365, 1366, 1367, 1368, 1369, 1370, 1371, 1372, 1373, 1374, 1375, 1376, 1377, 1378, 1379, 1380, 1381, 1382, 1383, 1384, 1385, 1386, 1387, 1388, 1389, 1390, 1391, 1392, 1393, 1394, 1395, 1396, 1397, 1398, 1399, 1400, 1401, 1402, 1403, 1404, 1405, 1406, 1407, 1408, 1409, 1410, 1411, 1412, 1413, 1414, 1415, 1416, 1417, 1418, 1419, 1420, 1421, 1422, 1423, 1424, 1425, 1426, 1427, 1428, 1429, 1430, 1431, 1432, 1433, 1434, 1435, 1436, 1437, 1438, 1439, 1440, 1441, 1442, 1443, 1444, 1445, 1446, 1447, 1448, 1449, 1450, 1451, 1452, 1453, 1454, 1455, 1456, 1457, 1458, 1459, 1460, 1461, 1462, 1463, 1464, 1465, 1466, 1467, 1468, 1469, 1470, 1471, 1472, 1473, 1474, 1475, 1476, 1477, 1478, 1479, 1480, 1481, 1482, 1483, 1484, 1485, 1486, 1487, 1488, 1489, 1490, 1491, 1492, 1493, 1494, 1495, 1496, 1497, 1498, 1499, 1500, 1501, 1502, 1503, 1504, 1505, 1506, 1507, 1508, 1509, 1510, 1511, 1512, 1513, 1514, 1515, 1516, 1517, 1518, 1519, 1520, 1521, 1522, 1523, 1524, 1525, 1526, 1527, 1528, 1529, 1530, 1531, 1532, 1533, 1534, 1535, 1536, 1537, 1538, 1539, 1540, 1541, 1542, 1543, 1544, 1545, 1546, 1547, 1548, 1549, 1550, 1551, 1552, 1553, 1554, 1555, 1556, 1557, 1558, 1559, 1560, 1561, 1562, 1563, 1564, 1565, 1566, 1567, 1568, 1569, 1570, 1571, 1572, 1573, 1574, 1575, 1576, 1577, 1578, 1579, 1580, 1581, 1582, 1583, 1584, 1585, 1586, 1587, 1588, 1589, 1590, 1591, 1592, 1593, 1594, 1595, 1596, 1597, 1598, 1599, 1600, 1601, 1602, 1603, 1604, 1605, 1606, 1607, 1608, 1609, 1610, 1611, 1612, 1613, 1614, 1615, 1616, 1617, 1618, 1619, 1620, 1621, 1622, 1623, 1624, 1625, 1626, 1627, 1628, 1629, 1630, 1631, 1632, 1633, 1634, 1635, 1636, 1637, 1638, 1639, 1640, 1641, 1642, 1643, 1644, 1645, 1646, 1647, 1648, 1649, 1650, 1651, 1652, 1653, 1654, 1655, 1656, 1657, 1658, 1659, 1660, 1661, 1662, 1663, 1664, 1665, 1666, 1667, 1668, 1669, 1670, 1671, 1672, 1673, 1674, 1675, 1676, 1677, 1678, 1679, 1680, 1681, 1682, 1683, 1684, 1685, 1686, 1687, 1688, 1689, 1690, 1691, 1692, 1693, 1694, 1695, 1696, 1697, 1698, 1699, 1700, 1701, 1702, 1703, 1704, 1705, 1706, 1707, 1708, 1709, 1710, 1711, 1712, 1713, 1714, 1715, 1716, 1717, 1718, 1719, 1720, 1721, 1722, 1723, 1724, 1725, 1726, 1727, 1728, 1729, 1730, 1731, 1732, 1733, 1734, 1735, 1736, 1737, 1738, 1739, 1740, 1741, 1742, 1743, 1744, 1745, 1746, 1747, 1748, 1749, 1750, 1751, 1752, 1753, 1754, 1755, 1756, 1757, 1758, 1759, 1760, 1761, 1762, 1763, 1764, 1765, 1766, 1767, 1768, 1769, 1770, 1771, 1772, 1773, 1774, 1775, 1776, 1777, 1778, 1779, 1780, 1781, 1782, 1783, 1784, 1785, 1786, 1787, 1788, 1789, 1790, 1791, 1792, 1793, 1794, 1795, 1796, 1797, 1798, 1799, 1800, 1801, 1802, 1803, 1804, 1805, 1806, 1807, 1808, 1809, 1810, 1811, 1812, 1813, 1814, 1815, 1816, 1817, 1818, 1819, 1820, 1821, 1822, 1823, 1824, 1825, 1826, 1827, 1828, 1829, 1830, 1831, 1832, 1833, 1834, 1835, 1836, 1837, 1838, 1839, 1840, 1841, 1842, 1843, 1844, 1845, 1846, 1847, 1848, 1849, 1850, 1851, 1852, 1853, 1854, 1855, 1856, 1857, 1858, 1859, 1860, 1861, 1862, 1863, 1864, 1865, 1866, 1867, 1868, 1869, 1870, 1871, 1872, 1873, 1874, 1875, 1876, 1877, 1878, 1879, 1880, 1881, 1882, 1883, 1884, 1885, 1886, 1887, 1888, 1889, 1890, 1891, 1892, 1893, 1894, 1895, 1896, 1897, 1898, 1899, 1900, 1901, 1902, 1903, 1904, 1905, 1906, 1907, 1908, 1909, 1910, 1911, 1912, 1913, 1914, 1915, 1916, 1917, 1918, 1919, 1920, 1921, 1922, 1923, 1924, 1925, 1926, 1927, 1928, 1929, 1930, 1931, 1932, 1933, 1934, 1935, 1936, 1937, 1938, 1939, 1940, 1941, 1942, 1943, 1944, 1945, 1946, 1947, 1948, 1949, 1950, 1951, 1952, 1953, 1954, 1955, 1956, 1957, 1958, 1959, 1960, 1961, 1962, 1963, 1964, 1965, 1966, 1967, 1968, 1969, 1970, 1971, 1972, 1973, 1974, 1975, 1976, 1977, 1978, 1979, 1980, 1981, 1982, 1983, 1984, 1985, 1986, 1987, 1988, 1989, 1990, 1991, 1992, 1993, 1994, 1995, 1996, 1997, 1998, 1999, 2000, 2001, 2002, 2003, 2004, 2005, 2006, 2007, 2008, 2009, 2010, 2011, 2012, 2013, 2014, 2015, 2016, 2017, 2018, 2019, 2020, 2021, 2022, 2023, 2024, 2025, 2026, 2027, 2028, 2029, 2030, 2031, 2032, 2033, 2034, 2035, 2036, 2037, 2038, 2039, 2040, 2041, 2042, 2043, 2044, 2045, 2046, 2047, 2048, 2049, 2050, 2051, 2052, 2053, 2054, 2055, 2056, 2057, 2058, 2059, 2060, 2061, 2062, 2063, 2064, 2065, 2066, 2067, 2068, 2069, 2070, 2071, 2072, 2073, 2074, 2075, 2076, 2077, 2078, 2079, 2080, 2081, 2082, 2083, 2084, 2085, 2086, 2087, 2088, 2089, 2090, 2091, 2092, 2093, 2094, 2095, 2096, 2097, 2098, 2099, 2100, 2101, 2102, 2103, 2104, 2105, 2106, 2107, 2108, 2109, 2110, 2111, 2112, 2113, 2114, 2115, 2116, 2117, 2118, 2119, 2120, 2121, 2122, 2123, 2124, 2125, 2126, 2127, 2128, 2129, 2130, 2131, 2132, 2133, 2134, 2135, 2136, 2137, 2138, 2139, 2140, 2141, 2142, 2143, 2144, 2145, 2146, 2147, 2148, 2149, 2150, 2151, 2152, 2153, 2154, 2155, 2156, 2157, 2158, 2159, 2160, 2161, 2162, 2163, 2164, 2165, 2166, 2167, 2168, 2169, 2170, 2171, 2172, 2173, 2174, 2175, 2176, 2177, 2178, 2179, 2180, 2181, 2182, 2183, 2184, 2185, 2186, 2187, 2188, 2189, 2190, 2191, 2192, 2193, 2194, 2195, 2196, 2197, 2198, 2199, 2200, 2201, 2202, 2203, 2204, 2205, 2206, 2207, 2208, 2209, 2210, 2211, 2212, 2213, 2214, 2215, 2216, 2217, 2218, 2219, 2220, 2221, 2222, 2223, 2224, 2225, 2226, 2227, 2228, 2229, 2230, 2231, 2232, 2233, 2234, 2235, 2236, 2237, 2238, 2239, 2240, 2241, 2242, 2243, 2244, 2245, 2246, 2247, 2248, 2249, 2250, 2251, 2252, 2253, 2254, 2255, 2256, 2257, 2258, 2259, 2260, 2261, 2262, 2263, 2264, 2265, 2266, 2267, 2268, 2269, 2270, 2271, 2272, 2273, 2274, 2275, 2276, 2277, 2278, 2279, 2280, 2281, 2282, 2283, 2284, 2285, 2286, 2287, 2288, 2289, 2290, 2291, 2292, 2293, 2294, 2295, 2296, 2297, 2298, 2299, 2300, 2301, 2302, 2303, 2304, 2305, 2306, 2307, 2308, 2309, 2310, 2311, 2312, 2313, 2314, 2315, 2316, 2317, 2318, 2319, 2320, 2321, 2322, 2323, 2324, 2325, 2326, 2327, 2328, 2329, 2330, 2331, 2332, 2333, 2334, 2335, 2336, 2337, 2338, 2339, 2340, 2341, 2342, 2343, 2344, 2345, 2346, 2347, 2348, 2349, 2350, 2351, 2352, 2353, 2354, 2355, 2356, 2357, 2358, 2359, 2360, 2361, 2362, 2363, 2364, 2365, 2366, 2367, 2368, 2369, 2370, 2371, 2372, 2373, 2374, 2375, 2376, 2377, 2378, 2379, 2380, 2381, 2382, 2383, 2384, 2385, 2386, 2387, 2388, 2389, 2390, 2391, 2392, 2393, 2394, 2395, 2396, 2397, 2398, 2399, 2400, 2401, 2402, 2403, 2404, 2405, 2406, 2407, 2408, 2409, 2410, 2411, 2412, 2413, 2414, 2415, 2416, 2417, 2418, 2419, 2420, 2421, 2422, 2423, 2424, 2425, 2426, 2427, 2428, 2429, 2430, 2431, 2432, 2433, 2434, 2435, 2436, 2437, 2438, 2439, 2440, 2441, 2442, 2443, 2444, 2445, 2446, 2447, 2448, 2449, 2450, 2451, 2452, 2453, 2454, 2455, 2456, 2457, 2458, 2459, 2460, 2461, 2462, 2463, 2464, 2465, 2466, 2467, 2468, 2469, 2470, 2471, 2472, 2473, 2474, 2475, 2476, 2477, 2478, 2479, 2480, 2481, 2482, 2483, 2484, 2485, 2486, 2487, 2488, 2489, 2490, 2491, 2492, 2493, 2494, 2495, 2496, 2497, 2498, 2499, 2500, 2501, 2502, 2503, 2504, 2505, 2506, 2507, 2508, 2509, 2510, 2511, 2512, 2513, 2514, 2515, 2516, 2517, 2518, 2519, 2520, 2521, 2522, 2523, 2524, 2525, 2526, 2527, 2528, 2529, 2530, 2531, 2532, 2533, 2534, 2535, 2536, 2537, 2538, 2539, 2540, 2541, 2542, 2543, 2544, 2545, 2546, 2547, 2548, 2549, 2550, 2551, 2552, 2553, 2554, 2555, 2556, 2557, 2558, 2559, 2560, 2561, 2562, 2563, 2564, 2565, 2566, 2567, 2568, 2569, 2570, 2571, 2572, 2573, 2574, 2575, 2576, 2577, 2578, 2579, 2580, 2581, 2582, 2583, 2584, 2585, 2586, 2587, 2588, 2589, 2590, 2591, 2592, 2593, 2594, 2595, 2596, 2597, 2598, 2599, 2600, 2601, 2602, 2603, 2604, 2605, 2606, 2607, 2608, 2609, 2610, 2611, 2612, 2613, 2614, 2615, 2616, 2617, 2618, 2619, 2620, 2621, 2622, 2623, 2624, 2625, 2626, 2627, 2628, 2629, 2630, 2631, 2632, 2633, 2634, 2635, 2636, 2637, 2638, 2639, 2640, 2641, 2642, 2643, 2644, 2645, 2646, 2647, 2648, 2649, 2650, 2651, 2652, 2653, 2654, 2655, 2656, 2657, 2658, 2659, 2660, 2661, 2662, 2663, 2664, 2665, 2666, 2667, 2668, 2669, 2670, 2671, 2672, 2673, 2674, 2675, 2676, 2677, 2678, 2679, 2680, 2681, 2682, 2683, 2684, 2685, 2686, 2687, 2688, 2689, 2690, 2691, 2692, 2693, 2694, 2695, 2696, 2697, 2698, 2699, 2700, 2701, 2702, 2703, 2704, 2705, 2706, 2707, 2708, 2709, 2710, 2711, 2712, 2713, 2714, 2715, 2716, 2717, 2718, 2719, 2720, 2721, 2722, 2723, 2724, 2725, 2726, 2727, 2728, 2729, 2730, 2731, 2732, 2733, 2734, 2735, 2736, 2737, 2738, 2739, 2740, 2741, 2742, 2743, 2744, 2745, 2746, 2747, 2748, 2749, 2750, 2751, 2752, 2753, 2754, 2755, 2756, 2757, 2758, 2759, 2760, 2761, 2762, 2763, 2764, 2765, 2766, 2767, 2768, 2769, 2770, 2771, 2772, 2773, 2774, 2775, 2776, 2777, 2778, 2779, 2780, 2781, 2782, 2783, 2784, 2785, 2786, 2787, 2788, 2789, 2790, 2791, 2792, 2793, 2794, 2795, 2796, 2797, 2798, 2799, 2800, 2801, 2802, 2803, 2804, 2805, 2806, 2807, 2808, 2809, 2810, 2811, 2812, 2813, 2814, 2815, 2816, 2817, 2818, 2819, 2820, 2821, 2822, 2823, 2824, 2825, 2826, 2827, 2828, 2829, 2830, 2831, 2832, 2833, 2834, 2835, 2836, 2837, 2838, 2839, 2840, 2841, 2842, 2843, 2844, 2845, 2846, 2847, 2848, 2849, 2850, 2851, 2852, 2853, 2854, 2855, 2856, 2857, 2858, 2859, 2860, 2861, 2862, 2863, 2864, 2865, 2866, 2867, 2868, 2869, 2870, 2871, 2872, 2873, 2874, 2875, 2876, 2877, 2878, 2879, 2880, 2881, 2882, 2883, 2884, 2885, 2886, 2887, 2888, 2889, 2890, 2891, 2892, 2893, 2894, 2895, 2896, 2897, 2898, 2899, 2900, 2901, 2902, 2903, 2904, 2905, 2906, 2907, 2908, 2909, 2910, 2911, 2912, 2913, 2914, 2915, 2916, 2917, 2918, 2919, 2920, 2921, 2922, 2923, 2924, 2925, 2926, 2927, 2928, 2929, 2930, 2931, 2932, 2933, 2934, 2935, 2936, 2937, 2938, 2939, 2940, 2941, 2942, 2943, 2944, 2945, 2946, 2947, 2948, 2949, 2950, 2951, 2952, 2953, 2954, 2955, 2956, 2957, 2958, 2959, 2960, 2961, 2962, 2963, 2964, 2965, 2966, 2967, 2968, 2969, 2970, 2971, 2972, 2973, 2974, 2975, 2976, 2977, 2978, 2979, 2980, 2981, 2982, 2983, 2984, 2985, 2986, 2987, 2988, 2989, 2990, 2991, 2992, 2993, 2994, 2995, 2996, 2997, 2998, 2999, 3000, 3001, 3002, 3003, 3004, 3005, 3006, 3007, 3008, 3009, 3010, 3011, 3012, 3013, 3014, 3015, 3016, 3017, 3018, 3019, 3020, 3021, 3022, 3023, 3024, 3025, 3026, 3027, 3028, 3029, 3030, 3031, 3032, 3033, 3034, 3035, 3036, 3037, 3038, 3039, 3040, 3041, 3042, 3043, 3044, 3045, 3046, 3047, 3048, 3049, 3050, 3051, 3052, 3053, 3054, 3055, 3056, 3057, 3058, 3059, 3060, 3061, 3062, 3063, 3064, 3065, 3066, 3067, 3068, 3069, 3070, 3071, 3072, 3073, 3074, 3075, 3076, 3077, 3078, 3079, 3080, 3081, 3082, 3083, 3084, 3085, 3086, 3087, 3088, 3089, 3090, 3091, 3092, 3093, 3094, 3095, 3096, 3097, 3098, 3099, 3100, 3101, 3102, 3103, 3104, 3105, 3106, 3107, 3108, 3109, 3110, 3111, 3112, 3113, 3114, 3115, 3116, 3117, 3118, 3119, 3120, 3121, 3122, 3123, 3124, 3125, 3126, 3127, 3128, 3129, 3130, 3131, 3132, 3133, 3134, 3135, 3136, 3137, 3138, 3139, 3140, 3141, 3142, 3143, 3144, 3145, 3146, 3147, 3148, 3149, 3150, 3151, 3152, 3153, 3154, 3155, 3156, 3157, 3158, 3159, 3160, 3161, 3162, 3163, 3164, 3165, 3166, 3167, 3168, 3169, 3170, 3171, 3172, 3173, 3174, 3175, 3176, 3177, 3178, 3179, 3180, 3181, 3182, 3183, 3184, 3185, 3186, 3187, 3188, 3189, 3190, 3191, 3192, 3193, 3194, 3195, 3196, 3197, 3198, 3199, 3200, 3201, 3202, 3203, 3204, 3205, 3206, 3207, 3208, 3209, 3210, 3211, 3212, 3213, 3214, 3215, 3216, 3217, 3218, 3219, 3220, 3221, 3222, 3223, 3224, 3225, 3226, 3227, 3228, 3229, 3230, 3231, 3232, 3233, 3234, 3235, 3236, 3237, 3238, 3239, 3240, 3241, 3242, 3243, 3244, 3245, 3246, 3247, 3248, 3249, 3250, 3251, 3252, 3253, 3254, 3255, 3256, 3257, 3258, 3259, 3260, 3261, 3262, 3263, 3264, 3265, 3266, 3267, 3268, 3269, 3270, 3271, 3272, 3273, 3274, 3275, 3276, 3277, 3278, 3279, 3280, 3281, 3282, 3283, 3284, 3285, 3286, 3287, 3288, 3289, 3290, 3291, 3292, 3293, 3294, 3295, 3296, 3297, 3298, 3299, 3300, 3301, 3302, 3303, 3304, 3305, 3306, 3307, 3308, 3309, 3310, 3311, 3312, 3313, 3314, 3315, 3316, 3317, 3318, 3319, 3320, 3321, 3322, 3323, 3324, 3325, 3326, 3327, 3328, 3329, 3330, 3331, 3332, 3333, 3334, 3335, 3336, 3337, 3338, 3339, 3340, 3341, 3342, 3343, 3344, 3345, 3346, 3347, 3348, 3349, 3350, 3351, 3352, 3353, 3354, 3355, 3356, 3357, 3358, 3359, 3360, 3361, 3362, 3363, 3364, 3365, 3366, 3367, 3368, 3369, 3370, 3371, 3372, 3373, 3374, 3375, 3376, 3377, 3378, 3379, 3380, 3381, 3382, 3383, 3384, 3385, 3386, 3387, 3388, 3389, 3390, 3391, 3392, 3393, 3394, 3395, 3396, 3397, 3398, 3399, 3400, 3401, 3402, 3403, 3404, 3405, 3406, 3407, 3408, 3409, 3410, 3411, 3412, 3413, 3414, 3415, 3416, 3417, 3418, 3419, 3420, 3421, 3422, 3423, 3424, 3425, 3426, 3427, 3428, 3429, 3430, 3431, 3432, 3433, 3434, 3435, 3436, 3437, 3438, 3439, 3440, 3441, 3442, 3443, 3444, 3445, 3446, 3447, 3448, 3449, 3450, 3451, 3452, 3453, 3454, 3455, 3456, 3457, 3458, 3459, 3460, 3461, 3462, 3463, 3464, 3465, 3466, 3467, 3468, 3469, 3470, 3471, 3472, 3473, 3474, 3475, 3476, 3477, 3478, 3479, 3480, 3481, 3482, 3483, 3484, 3485, 3486, 3487, 3488, 3489, 3490, 3491, 3492, 3493, 3494, 3495, 3496, 3497, 3498, 3499, 3500, 3501, 3502, 3503, 3504, 3505, 3506, 3507, 3508, 3509, 3510, 3511, 3512, 3513, 3514, 3515, 3516, 3517, 3518, 3519, 3520, 3521, 3522, 3523, 3524, 3525, 3526, 3527, 3528, 3529, 3530, 3531, 3532, 3533, 3534, 3535, 3536, 3537, 3538, 3539, 3540, 3541, 3542, 3543, 3544, 3545, 3546, 3547, 3548, 3549, 3550, 3551, 3552, 3553, 3554, 3555, 3556, 3557, 3558, 3559, 3560, 3561, 3562, 3563, 3564, 3565, 3566, 3567, 3568, 3569, 3570, 3571, 3572, 3573, 3574, 3575, 3576, 3577, 3578, 3579, 3580, 3581, 3582, 3583, 3584, 3585, 3586, 3587, 3588, 3589, 3590, 3591, 3592, 3593, 3594, 3595, 3596, 3597, 3598, 3599, 3600, 3601, 3602, 3603, 3604, 3605, 3606, 3607, 3608, 3609, 3610, 3611, 3612, 3613, 3614, 3615, 3616, 3617, 3618, 3619, 3620, 3621, 3622, 3623, 3624, 3625, 3626, 3627, 3628, 3629, 3630, 3631, 3632, 3633, 3634, 3635, 3636, 3637, 3638, 3639, 3640, 3641, 3642, 3643, 3644, 3645, 3646, 3647, 3648, 3649, 3650, 3651, 3652, 3653, 3654, 3655, 3656, 3657, 3658, 3659, 3660, 3661, 3662, 3663, 3664, 3665, 3666, 3667, 3668, 3669, 3670, 3671, 3672, 3673, 3674, 3675, 3676, 3677, 3678, 3679, 3680, 3681, 3682, 3683, 3684, 3685, 3686, 3687, 3688, 3689, 3690, 3691, 3692, 3693, 3694, 3695, 3696, 3697, 3698, 3699, 3700, 3701, 3702, 3703, 3704, 3705, 3706, 3707, 3708, 3709, 3710, 3711, 3712, 3713, 3714, 3715, 3716, 3717, 3718, 3719, 3720, 3721, 3722, 3723, 3724, 3725, 3726, 3727, 3728, 3729, 3730, 3731, 3732, 3733, 3734, 3735, 3736, 3737, 3738, 3739, 3740, 3741, 3742, 3743, 3744, 3745, 3746, 3747, 3748, 3749, 3750, 3751, 3752, 3753, 3754, 3755, 3756, 3757, 3758, 3759, 3760, 3761, 3762, 3763, 3764, 3765, 3766, 3767, 3768, 3769, 3770, 3771, 3772, 3773, 3774, 3775, 3776, 3777, 3778, 3779, 3780, 3781, 3782, 3783, 3784, 3785, 3786, 3787, 3788, 3789, 3790, 3791, 3792, 3793, 3794, 3795, 3796, 3797, 3798, 3799, 3800, 3801, 3802, 3803, 3804, 3805, 3806, 3807, 3808, 3809, 3810, 3811, 3812, 3813, 3814, 3815, 3816, 3817, 3818, 3819, 3820, 3821, 3822, 3823, 3824, 3825, 3826, 3827, 3828, 3829, 3830, 3831, 3832, 3833, 3834, 3835, 3836, 3837, 3838, 3839, 3840, 3841, 3842, 3843, 3844, 3845, 3846, 3847, 3848, 3849, 3850, 3851, 3852, 3853, 3854, 3855, 3856, 3857, 3858, 3859, 3860, 3861, 3862, 3863, 3864, 3865, 3866, 3867, 3868, 3869, 3870, 3871, 3872, 3873, 3874, 3875, 3876, 3877, 3878, 3879, 3880, 3881, 3882, 3883, 3884, 3885, 3886, 3887, 3888, 3889, 3890, 3891, 3892, 3893, 3894, 3895, 3896, 3897, 3898, 3899, 3900, 3901, 3902, 3903, 3904, 3905, 3906, 3907, 3908, 3909, 3910, 3911, 3912, 3913, 3914, 3915, 3916, 3917, 3918, 3919, 3920, 3921, 3922, 3923, 3924, 3925, 3926, 3927, 3928, 3929, 3930, 3931, 3932, 3933, 3934, 3935, 3936, 3937, 3938, 3939, 3940, 3941, 3942, 3943, 3944, 3945, 3946, 3947, 3948, 3949, 3950, 3951, 3952, 3953, 3954, 3955, 3956, 3957, 3958, 3959, 3960, 3961, 3962, 3963, 3964, 3965, 3966, 3967, 3968, 3969, 3970, 3971, 3972, 3973, 3974, 3975, 3976, 3977, 3978, 3979, 3980, 3981, 3982, 3983, 3984, 3985, 3986, 3987, 3988, 3989, 3990, 3991, 3992, 3993, 3994, 3995, 3996, 3997, 3998, 3999, 4000, 4001, 4002, 4003, 4004, 4005, 4006, 4007, 4008, 4009, 4010, 4011, 4012, 4013, 4014, 4015, 4016, 4017, 4018, 4019, 4020, 4021, 4022, 4023, 4024, 4025, 4026, 4027, 4028, 4029, 4030, 4031, 4032, 4033, 4034, 4035, 4036, 4037, 4038, 4039, 4040, 4041, 4042, 4043, 4044, 4045, 4046, 4047, 4048, 4049, 4050, 4051, 4052, 4053, 4054, 4055, 4056, 4057, 4058, 4059, 4060, 4061, 4062, 4063, 4064, 4065, 4066, 4067, 4068, 4069, 4070, 4071, 4072, 4073, 4074, 4075, 4076, 4077, 4078, 4079, 4080, 4081, 4082, 4083, 4084, 4085, 4086, 4087, 4088, 4089, 4090, 4091, 4092, 4093, 4094, 4095, 4096, 4097, 4098, 4099, 4100, 4101, 4102, 4103, 4104, 4105, 4106, 4107, 4108, 4109, 4110, 4111, 4112, 4113, 4114, 4115, 4116, 4117, 4118, 4119, 4120, 4121, 4122, 4123, 4124, 4125, 4126, 4127, 4128, 4129, 4130, 4131, 4132, 4133, 4134, 4135, 4136, 4137, 4138, 4139, 4140, 4141, 4142, 4143, 4144, 4145, 4146, 4147, 4148, 4149, 4150, 4151, 4152, 4153, 4154, 4155, 4156, 4157, 4158, 4159, 4160, 4161, 4162, 4163, 4164, 4165, 4166, 4167, 4168, 4169, 4170, 4171, 4172, 4173, 4174, 4175, 4176, 4177, 4178, 4179, 4180, 4181, 4182, 4183, 4184, 4185, 4186, 4187, 4188, 4189, 4190, 4191, 4192, 4193, 4194, 4195, 4196, 4197, 4198, 4199, 4200, 4201, 4202, 4203, 4204, 4205, 4206, 4207, 4208, 4209, 4210, 4211, 4212, 4213, 4214, 4215, 4216, 4217, 4218, 4219, 4220, 4221, 4222, 4223, 4224, 4225, 4226, 4227, 4228, 4229, 4230, 4231, 4232, 4233, 4234, 4235, 4236, 4237, 4238, 4239, 4240, 4241, 4242, 4243, 4244, 4245, 4246, 4247, 4248, 4249, 4250, 4251, 4252, 4253, 4254, 4255, 4256, 4257, 4258, 4259, 4260, 4261, 4262, 4263, 4264, 4265, 4266, 4267, 4268, 4269, 4270, 4271, 4272, 4273, 4274, 4275, 4276, 4277, 4278, 4279, 4280, 4281, 4282, 4283, 4284, 4285, 4286, 4287, 4288, 4289, 4290, 4291, 4292, 4293, 4294, 4295, 4296, 4297, 4298, 4299, 4300, 4301, 4302, 4303, 4304, 4305, 4306, 4307, 4308, 4309, 4310, 4311, 4312, 4313, 4314, 4315, 4316, 4317, 4318, 4319, 4320, 4321, 4322, 4323, 4324, 4325, 4326, 4327, 4328, 4329, 4330, 4331, 4332, 4333, 4334, 4335, 4336, 4337, 4338, 4339, 4340, 4341, 4342, 4343, 4344, 4345, 4346, 4347, 4348, 4349, 4350, 4351, 4352, 4353, 4354, 4355, 4356, 4357, 4358, 4359, 4360, 4361, 4362, 4363, 4364, 4365, 4366, 4367, 4368, 4369, 4370, 4371, 4372, 4373, 4374, 4375, 4376, 4377, 4378, 4379, 4380, 4381, 4382, 4383, 4384, 4385, 4386, 4387, 4388, 4389, 4390, 4391, 4392, 4393, 4394, 4395, 4396, 4397, 4398, 4399, 4400, 4401, 4402, 4403, 4404, 4405, 4406, 4407, 4408, 4409, 4410, 4411, 4412, 4413, 4414, 4415, 4416, 4417, 4418, 4419, 4420, 4421, 4422, 4423, 4424, 4425, 4426, 4427, 4428, 4429, 4430, 4431, 4432, 4433, 4434, 4435, 4436, 4437, 4438, 4439, 4440, 4441, 4442, 4443, 4444, 4445, 4446, 4447, 4448, 4449, 4450, 4451, 4452, 4453, 4454, 4455, 4456, 4457, 4458, 4459, 4460, 4461, 4462, 4463, 4464, 4465, 4466, 4467, 4468, 4469, 4470, 4471, 4472, 4473, 4474, 4475, 4476, 4477, 4478, 4479, 4480, 4481, 4482, 4483, 4484, 4485, 4486, 4487, 4488, 4489, 4490, 4491, 4492, 4493, 4494, 4495, 4496, 4497, 4498, 4499, 4500, 4501, 4502, 4503, 4504, 4505, 4506, 4507, 4508, 4509, 4510, 4511, 4512, 4513, 4514, 4515, 4516, 4517, 4518, 4519, 4520, 4521, 4522, 4523, 4524, 4525, 4526, 4527, 4528, 4529, 4530, 4531, 4532, 4533, 4534, 4535, 4536, 4537, 4538, 4539, 4540, 4541, 4542, 4543, 4544, 4545, 4546, 4547, 4548, 4549, 4550, 4551, 4552, 4553, 4554, 4555, 4556, 4557, 4558, 4559, 4560, 4561, 4562, 4563, 4564, 4565, 4566, 4567, 4568, 4569, 4570, 4571, 4572, 4573, 4574, 4575, 4576, 4577, 4578, 4579, 4580, 4581, 4582, 4583, 4584, 4585, 4586, 4587, 4588, 4589, 4590, 4591, 4592, 4593, 4594, 4595, 4596, 4597, 4598, 4599, 4600, 4601, 4602, 4603, 4604, 4605, 4606, 4607, 4608, 4609, 4610, 4611, 4612, 4613, 4614, 4615, 4616, 4617, 4618, 4619, 4620, 4621, 4622, 4623, 4624, 4625, 4626, 4627, 4628, 4629, 4630, 4631, 4632, 4633, 4634, 4635, 4636, 4637, 4638, 4639, 4640, 4641, 4642, 4643, 4644, 4645, 4646, 4647, 4648, 4649, 4650, 4651, 4652, 4653, 4654, 4655, 4656, 4657, 4658, 4659, 4660, 4661, 4662, 4663, 4664, 4665, 4666, 4667, 4668, 4669, 4670, 4671, 4672, 4673, 4674, 4675, 4676, 4677, 4678, 4679, 4680, 4681, 4682, 4683, 4684, 4685, 4686, 4687, 4688, 4689, 4690, 4691, 4692, 4693, 4694, 4695, 4696, 4697, 4698, 4699, 4700, 4701, 4702, 4703, 4704, 4705, 4706, 4707, 4708, 4709, 4710, 4711, 4712, 4713, 4714, 4715, 4716, 4717, 4718, 4719, 4720, 4721, 4722, 4723, 4724, 4725, 4726, 4727, 4728, 4729, 4730, 4731, 4732, 4733, 4734, 4735, 4736, 4737, 4738, 4739, 4740, 4741, 4742, 4743, 4744, 4745, 4746, 4747, 4748, 4749, 4750, 4751, 4752, 4753, 4754, 4755, 4756, 4757, 4758, 4759, 4760, 4761, 4762, 4763, 4764, 4765, 4766, 4767, 4768, 4769, 4770, 4771, 4772, 4773, 4774, 4775, 4776, 4777, 4778, 4779, 4780, 4781, 4782, 4783, 4784, 4785, 4786, 4787, 4788, 4789, 4790, 4791, 4792, 4793, 4794, 4795, 4796, 4797, 4798, 4799, 4800, 4801, 4802, 4803, 4804, 4805, 4806, 4807, 4808, 4809, 4810, 4811, 4812, 4813, 4814, 4815, 4816, 4817, 4818, 4819, 4820, 4821, 4822, 4823, 4824, 4825, 4826, 4827, 4828, 4829, 4830, 4831, 4832, 4833, 4834, 4835, 4836, 4837, 4838, 4839, 4840, 4841, 4842, 4843, 4844, 4845, 4846, 4847, 4848, 4849, 4850, 4851, 4852, 4853, 4854, 4855, 4856, 4857, 4858, 4859, 4860, 4861, 4862, 4863, 4864, 4865, 4866, 4867, 4868, 4869, 4870, 4871, 4872, 4873, 4874, 4875, 4876, 4877, 4878, 4879, 4880, 4881, 4882, 4883, 4884, 4885, 4886, 4887, 4888, 4889, 4890, 4891, 4892, 4893, 4894, 4895, 4896, 4897, 4898, 4899, 4900, 4901, 4902, 4903, 4904, 4905, 4906, 4907, 4908, 4909, 4910, 4911, 4912, 4913, 4914, 4915, 4916, 4917, 4918, 4919, 4920, 4921, 4922, 4923, 4924, 4925, 4926, 4927, 4928, 4929, 4930, 4931, 4932, 4933, 4934, 4935, 4936, 4937, 4938, 4939, 4940, 4941, 4942, 4943, 4944, 4945, 4946, 4947, 4948, 4949, 4950, 4951, 4952, 4953, 4954, 4955, 4956, 4957, 4958, 4959, 4960, 4961, 4962, 4963, 4964, 4965, 4966, 4967, 4968, 4969, 4970, 4971, 4972, 4973, 4974, 4975, 4976, 4977, 4978, 4979, 4980, 4981, 4982, 4983, 4984, 4985, 4986, 4987, 4988, 4989, 4990, 4991, 4992, 4993, 4994, 4995, 4996, 4997, 4998, 4999 and 5000%.

In some embodiments, the stabilised halocarbon compound containing compositions of the invention are stabilised via the formation of an emulsion, or a micelle, or a liposome, or a hydrogel or an oil solution.

In a preferred embodiment, the liposome is not derived from a red marine macroalgae cell, or a yeast cell.

In some embodiments, the stabilised halocarbon compound containing compositions of the invention are stabilised via spray drying a stabilised emulsion or a stabilised micelle, or a stabilised liposome, or a stabilised hydrogel.

In a preferred embodiment, the stabilised halocarbon compound containing compositions of the invention do not comprise methylcellulose, or cyclodextrin.

In some embodiments, the stabilised halocarbon compound containing compositions of the invention are stabilised via encapsulation.

In a preferred embodiment, the stabilised halocarbon compound containing compositions of the invention which are stabilised via encapsulation, comprise an encapsulating agent which does not comprise polyethylenemaleic anhydride copolymer.

In some embodiments, the stabilised halocarbon compound containing compositions of the invention are stabilised via the formation of a solution of the halocarbon compound in an oil, optionally in the presence of an alcohol. Most preferably the oil is a medium chain triglyceride (MCT). Most preferably the alcohol is ethanol.

In some embodiments, the stabilised halocarbon compound containing compositions of the invention are emulsions, or micelles, or liposomes, or hydrogels, or oil solutions, comprising a halocarbon compound within the emulsion, or micelle, or liposome, or hydrogel or oil solution.

In some embodiments, the stabilised halocarbon compound containing compositions of the invention are dry powders, comprising a halocarbon compound.

In a preferred embodiment, the stabilised halocarbon compound containing compositions of the invention which are dry powders comprising a halocarbon compound, do not comprise methylcellulose, or polyethylenemaleic anhydride copolymer, or cyclodextrin or a cyclic oligosaccharide.

In some embodiments, the stabilised halocarbon compound containing compositions of the invention are dry powders, comprising an encapsulated halocarbon compound.

In a preferred embodiment, the stabilised halocarbon compound containing compositions of the invention which are dry powders, comprising an encapsulated halocarbon compound, do not comprise methylcellulose, or cyclodextrin, or polyethylenemaleic anhydride copolymer.

In some embodiments, the compositions of the invention are in the form of a solid pellet, pill, or tablet; optionally wherein the solid pellet, pill, or tablet is suitable for incorporation into a bolus; preferably wherein the solid pellet, pill, or tablet is adapted to provide in vivo slow-release of the halocarbon compound in the rumen of a ruminant; preferably wherein the composition comprises gelatin; preferably wherein the halocarbon compound is bromoform.

In one preferred embodiment, the stabilised halocarbon compound containing composition of the invention is an oil solution comprising a halocarbon compound (preferably bromoform) and an oil (preferably MCT); optionally further comprising an alcohol (preferably ethanol).

In one preferred embodiment, the stabilised halocarbon compound containing composition of the invention is an emulsion comprising bromoform within the emulsion.

In another preferred embodiment, the stabilised halocarbon compound containing composition of the invention is a dry powder, comprising bromoform.

In another preferred embodiment, the stabilised halocarbon compound containing composition of the invention is a dry powder, comprising encapsulated bromoform.

In some embodiments, the dry powders comprising a halocarbon compound (preferably bromoform) are prepared from an emulsion, or micelle, or liposome, or hydrogel, comprising a halocarbon compound (preferably bromoform).

In a preferred embodiment, the dry powders comprising a halocarbon compound (preferably bromoform), prepared from an emulsion, or micelle, or liposome, or hydrogel, comprising a halocarbon compound (preferably bromoform), do not comprise methylcellulose, or cyclodextrin, or polyethylenemaleic anhydride copolymer.

In some embodiments, the dry powders comprising an encapsulated halocarbon compound (preferably bromoform) are prepared from an emulsion or micelle, or liposome, or hydrogel, comprising a halocarbon compound (preferably bromoform).

In a preferred embodiment, the dry powders comprising an encapsulated halocarbon compound (preferably bromoform), prepared from an emulsion or micelle, or liposome, or hydrogel, comprising a halocarbon compound (preferably bromoform), do not comprise methylcellulose, or cyclodextrin, or polyethylenemaleic anhydride copolymer.

In some embodiments, the emulsions, or the micelles, or the liposomes, or the hydrogels, or the dry powders, or the oil solutions of the invention further comprise one or more proteins (including globular proteins such as those present in milk), peptides, oligopeptides or amino acids. Preferred peptides include, without limitation, hydrolyzed collagen, collagen hydrolysate, gelatine hydrolysate, hydrolyzed gelatine, or gelatin.

In some embodiments, the emulsions, or the micelles, or the liposomes, or the hydrogels, or the dry powders, or the oil solutions of the invention further comprise one or more carbohydrates, saccharides, disaccharides, oligosaccharides, polysaccharides, starches or celluloses. Preferred polysaccharides include, without limitation, xanthan gum (used interchangeably herein with the term xanthum gum), or guar gum.

In some embodiments, the emulsions, or the micelles, or the liposomes, or the hydrogels, or the dry powders, or the oil solutions of the invention further comprise one or more lipids, fatty acids, waxes, oils, sterols, fat-soluble vitamins (such as vitamins A, D, E, and K), monoglycerides, diglycerides, triglycerides, phospholipids, glycerolipids, sphingolipids, saccharolipids, polyketides, sterol lipids or prenol lipids. Preferred triglycerides include, without limitation, medium chain triglycerides (MCT), or caprylic/capric triglyceride.

In some embodiments, the emulsions, or the micelles, or the liposomes, or the hydrogels, or the dry powders, or the oil solutions of the invention further comprise one or more emulsifying agents, glycerol, sorbitol, lecithins, glycerophospholipids, phosphatidylcholines, phosphatidylethanolamines, phosphatidylinositols, phosphatidylserines, or phosphatidic acids.

In some embodiments, the emulsions, or the micelles, or the liposomes, or the hydrogels, or the dry powders, or the oil solutions of the invention further comprise one or more surfactants, food acids, diacetyl tartaric acid esters of mono- and diglycerides, or detergents. Preferred surfactants include, without limitation, polysorbates such as Tween 20, Tween 40, Tween 60, or Tween 80, or analogues or equivalents thereof.

In some embodiments, the emulsions, or the micelles, or the liposomes, or the hydrogels, or the dry powders, or the oil solutions of the invention further comprise one or more encapsulating agents, alkyl celluloses, ethyl celluloses, polyvinyl alcohols, gelatins, carageenans, hydrogels, alginates or alginate salts.

In some embodiments, the emulsions, or the micelles, or the liposomes, or the hydrogels, or the dry powders, or the oil solutions of the invention further comprise one or more edible or biocompatible polymers.

In some embodiments, the emulsions, or the micelles, or the liposomes, or the hydrogels, or the dry powders, or the oil solutions of the invention comprise fresh milk, and/or whole milk powder, and/or ethanol, and/or water.

In some embodiments, the emulsions, or the micelles, or the liposomes, or the hydrogels, or the dry powders, or the oil solutions of the invention comprise one or more active agents.

In some embodiments, the emulsions, or the micelles, or the liposomes, or the hydrogels, or the dry powders, or the oil solutions of the invention comprise a halocarbon compound (preferably bromoform) at a concentration selected from a concentration falling within the range of about 1 ppm to about 5000 ppm.

In some embodiments, the emulsions, or the micelles, or the liposomes, or the hydrogels, or the dry powders, or the oil solutions of the invention comprise a halocarbon compound (preferably bromoform) at a concentration selected from the group consisting of a concentration of about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295, 296, 297, 298, 299, 300, 301, 302, 303, 304, 305, 306, 307, 308, 309, 310, 311, 312, 313, 314, 315, 316, 317, 318, 319, 320, 321, 322, 323, 324, 325, 326, 327, 328, 329, 330, 331, 332, 334, 335, 336, 337, 338, 339, 340, 341, 342, 343, 344, 345, 346, 347, 348, 349, 350, 351, 352, 353, 354, 355, 356, 357, 358, 359, 360, 361, 362, 363, 364, 365, 366, 367, 368, 369, 370, 371, 372, 373, 374, 375, 376, 377, 378, 379, 380, 381, 382, 383, 384, 385, 386, 387, 388, 389, 390, 391, 392, 393, 394, 395, 396, 397, 398, 399, 400, 401, 402, 403, 404, 405, 406, 407, 408, 409, 410, 411, 412, 413, 414, 415, 416, 417, 418, 419, 420, 421, 422, 423, 424, 425, 426, 427, 428, 429, 430, 431, 432, 433, 434, 435, 436, 437, 438, 439, 440, 441, 442, 443, 444, 445, 446, 447, 448, 449, 450, 451, 452, 453, 454, 455, 456, 457, 458, 459, 460, 461, 462, 463, 464, 465, 466, 467, 468, 469, 470, 471, 472, 473, 474, 475, 476, 477, 478, 479, 480, 481, 482, 483, 484, 485, 486, 487, 488, 489, 490, 491, 492, 493, 494, 495, 496, 497, 498, 499, 500, 501, 502, 503, 504, 505, 506, 507, 508, 509, 510, 511, 512, 513, 514, 515, 516, 517, 518, 519, 520, 521, 522, 523, 524, 525, 526, 527, 528, 529, 530, 531, 532, 533, 534, 535, 536, 537, 538, 539, 540, 541, 542, 543, 544, 545, 546, 547, 548, 549, 550, 551, 552, 553, 554, 555, 556, 557, 558, 559, 560, 561, 562, 563, 564, 565, 566, 567, 568, 569, 570, 571, 572, 573, 574, 575, 576, 577, 578, 579, 580, 581, 582, 583, 584, 585, 586, 587, 588, 589, 590, 591, 592, 593, 594, 595, 596, 597, 598, 599, 600, 601, 602, 603, 604, 605, 606, 607, 608, 609, 610, 611, 612, 613, 614, 615, 616, 617, 618, 619, 620, 621, 622, 623, 624, 625, 626, 627, 628, 629, 630, 631, 632, 633, 634, 635, 636, 637, 638, 639, 640, 641, 642, 643, 644, 645, 646, 647, 648, 649, 650, 651, 652, 653, 654, 655, 656, 657, 658, 659, 660, 661, 662, 663, 664, 665, 666, 667, 668, 669, 670, 671, 672, 673, 674, 675, 676, 677, 678, 679, 680, 681, 682, 683, 684, 685, 686, 687, 688, 689, 690, 691, 692, 693, 694, 695, 696, 697, 698, 699, 700, 701, 702, 703, 704, 705, 706, 707, 708, 709, 710, 711, 712, 713, 714, 715, 716, 717, 718, 719, 720, 721, 722, 723, 724, 725, 726, 727, 728, 729, 730, 731, 732, 733, 734, 735, 736, 737, 738, 739, 740, 741, 742, 743, 744, 745, 746, 747, 748, 749, 750, 751, 752, 753, 754, 755, 756, 757, 758, 759, 760, 761, 762, 763, 764, 765, 766, 767, 768, 769, 770, 771, 772, 773, 774, 775, 776, 777, 778, 779, 780, 781, 782, 783, 784, 785, 786, 787, 788, 789, 790, 791, 792, 793, 794, 795, 796, 797, 798, 799, 800, 801, 802, 803, 804, 805, 806, 807, 808, 809, 810, 811, 812, 813, 814, 815, 816, 817, 818, 819, 820, 821, 822, 823, 824, 825, 826, 827, 828, 829, 830, 831, 832, 833, 834, 835, 836, 837, 838, 839, 840, 841, 842, 843, 844, 845, 846, 847, 848, 849, 850, 851, 852, 853, 854, 855, 856, 857, 858, 859, 860, 861, 862, 863, 864, 865, 866, 867, 868, 869, 870, 871, 872, 873, 874, 875, 876, 877, 878, 879, 880, 881, 882, 883, 884, 885, 886, 887, 888, 889, 890, 891, 892, 893, 894, 895, 896, 897, 898, 899, 900, 901, 902, 903, 904, 905, 906, 907, 908, 909, 910, 911, 912, 913, 914, 915, 916, 917, 918, 919, 920, 921, 922, 923, 924, 925, 926, 927, 928, 929, 930, 931, 932, 933, 934, 935, 936, 937, 938, 939, 940, 941, 942, 943, 944, 945, 946, 947, 948, 949, 950, 951, 952, 953, 954, 955, 956, 957, 958, 959, 960, 961, 962, 963, 964, 965, 966, 967, 968, 969, 970, 971, 972, 973, 974, 975, 976, 977, 978, 979, 980, 981, 982, 983, 984, 985, 986, 987, 988, 989, 990, 991, 992, 993, 994, 995, 996, 997, 998, 999, 1000, 1001, 1002, 1003, 1004, 1005, 1006, 1007, 1008, 1009, 1010, 1011, 1012, 1013, 1014, 1015, 1016, 1017, 1018, 1019, 1020, 1021, 1022, 1023, 1024, 1025, 1026, 1027, 1028, 1029, 1030, 1031, 1032, 1033, 1034, 1035, 1036, 1037, 1038, 1039, 1040, 1041, 1042, 1043, 1044, 1045, 1046, 1047, 1048, 1049, 1050, 1051, 1052, 1053, 1054, 1055, 1056, 1057, 1058, 1059, 1060, 1061, 1062, 1063, 1064, 1065, 1066, 1067, 1068, 1069, 1070, 1071, 1072, 1073, 1074, 1075, 1076, 1077, 1078, 1079, 1080, 1081, 1082, 1083, 1084, 1085, 1086, 1087, 1088, 1089, 1090, 1091, 1092, 1093, 1094, 1095, 1096, 1097, 1098, 1099, 1100, 1101, 1102, 1103, 1104, 1105, 1106, 1107, 1108, 1109, 1110, 1111, 1112, 1113, 1114, 1115, 1116, 1117, 1118, 1119, 1120, 1121, 1122, 1123, 1124, 1125, 1126, 1127, 1128, 1129, 1130, 1131, 1132, 1133, 1134, 1135, 1136, 1137, 1138, 1139, 1140, 1141, 1142, 1143, 1144, 1145, 1146, 1147, 1148, 1149, 1150, 1151, 1152, 1153, 1154, 1155, 1156, 1157, 1158, 1159, 1160, 1161, 1162, 1163, 1164, 1165, 1166, 1167, 1168, 1169, 1170, 1171, 1172, 1173, 1174, 1175, 1176, 1177, 1178, 1179, 1180, 1181, 1182, 1183, 1184, 1185, 1186, 1187, 1188, 1189, 1190, 1191, 1192, 1193, 1194, 1195, 1196, 1197, 1198, 1199, 1200, 1201, 1202, 1203, 1204, 1205, 1206, 1207, 1208, 1209, 1210, 1211, 1212, 1213, 1214, 1215, 1216, 1217, 1218, 1219, 1220, 1221, 1222, 1223, 1224, 1225, 1226, 1227, 1228, 1229, 1230, 1231, 1232, 1233, 1234, 1235, 1236, 1237, 1238, 1239, 1240, 1241, 1242, 1243, 1244, 1245, 1246, 1247, 1248, 1249, 1250, 1251, 1252, 1253, 1254, 1255, 1256, 1257, 1258, 1259, 1260, 1261, 1262, 1263, 1264, 1265, 1266, 1267, 1268, 1269, 1270, 1271, 1272, 1273, 1274, 1275, 1276, 1277, 1278, 1279, 1280, 1281, 1282, 1283, 1284, 1285, 1286, 1287, 1288, 1289, 1290, 1291, 1292, 1293, 1294, 1295, 1296, 1297, 1298, 1299, 1300, 1301, 1302, 1303, 1304, 1305, 1306, 1307, 1308, 1309, 1310, 1311, 1312, 1313, 1314, 1315, 1316, 1317, 1318, 1319, 1320, 1321, 1322, 1323, 1324, 1325, 1326, 1327, 1328, 1329, 1330, 1331, 1332, 1333, 1334, 1335, 1336, 1337, 1338, 1339, 1340, 1341, 1342, 1343, 1344, 1345, 1346, 1347, 1348, 1349, 1350, 1351, 1352, 1353, 1354, 1355, 1356, 1357, 1358, 1359, 1360, 1361, 1362, 1363, 1364, 1365, 1366, 1367, 1368, 1369, 1370, 1371, 1372, 1373, 1374, 1375, 1376, 1377, 1378, 1379, 1380, 1381, 1382, 1383, 1384, 1385, 1386, 1387, 1388, 1389, 1390, 1391, 1392, 1393, 1394, 1395, 1396, 1397, 1398, 1399, 1400, 1401, 1402, 1403, 1404, 1405, 1406, 1407, 1408, 1409, 1410, 1411, 1412, 1413, 1414, 1415, 1416, 1417, 1418, 1419, 1420, 1421, 1422, 1423, 1424, 1425, 1426, 1427, 1428, 1429, 1430, 1431, 1432, 1433, 1434, 1435, 1436, 1437, 1438, 1439, 1440, 1441, 1442, 1443, 1444, 1445, 1446, 1447, 1448, 1449, 1450, 1451, 1452, 1453, 1454, 1455, 1456, 1457, 1458, 1459, 1460, 1461, 1462, 1463, 1464, 1465, 1466, 1467, 1468, 1469, 1470, 1471, 1472, 1473, 1474, 1475, 1476, 1477, 1478, 1479, 1480, 1481, 1482, 1483, 1484, 1485, 1486, 1487, 1488, 1489, 1490, 1491, 1492, 1493, 1494, 1495, 1496, 1497, 1498, 1499, 1500, 1501, 1502, 1503, 1504, 1505, 1506, 1507, 1508, 1509, 1510, 1511, 1512, 1513, 1514, 1515, 1516, 1517, 1518, 1519, 1520, 1521, 1522, 1523, 1524, 1525, 1526, 1527, 1528, 1529, 1530, 1531, 1532, 1533, 1534, 1535, 1536, 1537, 1538, 1539, 1540, 1541, 1542, 1543, 1544, 1545, 1546, 1547, 1548, 1549, 1550, 1551, 1552, 1553, 1554, 1555, 1556, 1557, 1558, 1559, 1560, 1561, 1562, 1563, 1564, 1565, 1566, 1567, 1568, 1569, 1570, 1571, 1572, 1573, 1574, 1575, 1576, 1577, 1578, 1579, 1580, 1581, 1582, 1583, 1584, 1585, 1586, 1587, 1588, 1589, 1590, 1591, 1592, 1593, 1594, 1595, 1596, 1597, 1598, 1599, 1600, 1601, 1602, 1603, 1604, 1605, 1606, 1607, 1608, 1609, 1610, 1611, 1612, 1613, 1614, 1615, 1616, 1617, 1618, 1619, 1620, 1621, 1622, 1623, 1624, 1625, 1626, 1627, 1628, 1629, 1630, 1631, 1632, 1633, 1634, 1635, 1636, 1637, 1638, 1639, 1640, 1641, 1642, 1643, 1644, 1645, 1646, 1647, 1648, 1649, 1650, 1651, 1652, 1653, 1654, 1655, 1656, 1657, 1658, 1659, 1660, 1661, 1662, 1663, 1664, 1665, 1666, 1667, 1668, 1669, 1670, 1671, 1672, 1673, 1674, 1675, 1676, 1677, 1678, 1679, 1680, 1681, 1682, 1683, 1684, 1685, 1686, 1687, 1688, 1689, 1690, 1691, 1692, 1693, 1694, 1695, 1696, 1697, 1698, 1699, 1700, 1701, 1702, 1703, 1704, 1705, 1706, 1707, 1708, 1709, 1710, 1711, 1712, 1713, 1714, 1715, 1716, 1717, 1718, 1719, 1720, 1721, 1722, 1723, 1724, 1725, 1726, 1727, 1728, 1729, 1730, 1731, 1732, 1733, 1734, 1735, 1736, 1737, 1738, 1739, 1740, 1741, 1742, 1743, 1744, 1745, 1746, 1747, 1748, 1749, 1750, 1751, 1752, 1753, 1754, 1755, 1756, 1757, 1758, 1759, 1760, 1761, 1762, 1763, 1764, 1765, 1766, 1767, 1768, 1769, 1770, 1771, 1772, 1773, 1774, 1775, 1776, 1777, 1778, 1779, 1780, 1781, 1782, 1783, 1784, 1785, 1786, 1787, 1788, 1789, 1790, 1791, 1792, 1793, 1794, 1795, 1796, 1797, 1798, 1799, 1800, 1801, 1802, 1803, 1804, 1805, 1806, 1807, 1808, 1809, 1810, 1811, 1812, 1813, 1814, 1815, 1816, 1817, 1818, 1819, 1820, 1821, 1822, 1823, 1824, 1825, 1826, 1827, 1828, 1829, 1830, 1831, 1832, 1833, 1834, 1835, 1836, 1837, 1838, 1839, 1840, 1841, 1842, 1843, 1844, 1845, 1846, 1847, 1848, 1849, 1850, 1851, 1852, 1853, 1854, 1855, 1856, 1857, 1858, 1859, 1860, 1861, 1862, 1863, 1864, 1865, 1866, 1867, 1868, 1869, 1870, 1871, 1872, 1873, 1874, 1875, 1876, 1877, 1878, 1879, 1880, 1881, 1882, 1883, 1884, 1885, 1886, 1887, 1888, 1889, 1890, 1891, 1892, 1893, 1894, 1895, 1896, 1897, 1898, 1899, 1900, 1901, 1902, 1903, 1904, 1905, 1906, 1907, 1908, 1909, 1910, 1911, 1912, 1913, 1914, 1915, 1916, 1917, 1918, 1919, 1920, 1921, 1922, 1923, 1924, 1925, 1926, 1927, 1928, 1929, 1930, 1931, 1932, 1933, 1934, 1935, 1936, 1937, 1938, 1939, 1940, 1941, 1942, 1943, 1944, 1945, 1946, 1947, 1948, 1949, 1950, 1951, 1952, 1953, 1954, 1955, 1956, 1957, 1958, 1959, 1960, 1961, 1962, 1963, 1964, 1965, 1966, 1967, 1968, 1969, 1970, 1971, 1972, 1973, 1974, 1975, 1976, 1977, 1978, 1979, 1980, 1981, 1982, 1983, 1984, 1985, 1986, 1987, 1988, 1989, 1990, 1991, 1992, 1993, 1994, 1995, 1996, 1997, 1998, 1999, 2000, 2001, 2002, 2003, 2004, 2005, 2006, 2007, 2008, 2009, 2010, 2011, 2012, 2013, 2014, 2015, 2016, 2017, 2018, 2019, 2020, 2021, 2022, 2023, 2024, 2025, 2026, 2027, 2028, 2029, 2030, 2031, 2032, 2033, 2034, 2035, 2036, 2037, 2038, 2039, 2040, 2041, 2042, 2043, 2044, 2045, 2046, 2047, 2048, 2049, 2050, 2051, 2052, 2053, 2054, 2055, 2056, 2057, 2058, 2059, 2060, 2061, 2062, 2063, 2064, 2065, 2066, 2067, 2068, 2069, 2070, 2071, 2072, 2073, 2074, 2075, 2076, 2077, 2078, 2079, 2080, 2081, 2082, 2083, 2084, 2085, 2086, 2087, 2088, 2089, 2090, 2091, 2092, 2093, 2094, 2095, 2096, 2097, 2098, 2099, 2100, 2101, 2102, 2103, 2104, 2105, 2106, 2107, 2108, 2109, 2110, 2111, 2112, 2113, 2114, 2115, 2116, 2117, 2118, 2119, 2120, 2121, 2122, 2123, 2124, 2125, 2126, 2127, 2128, 2129, 2130, 2131, 2132, 2133, 2134, 2135, 2136, 2137, 2138, 2139, 2140, 2141, 2142, 2143, 2144, 2145, 2146, 2147, 2148, 2149, 2150, 2151, 2152, 2153, 2154, 2155, 2156, 2157, 2158, 2159, 2160, 2161, 2162, 2163, 2164, 2165, 2166, 2167, 2168, 2169, 2170, 2171, 2172, 2173, 2174, 2175, 2176, 2177, 2178, 2179, 2180, 2181, 2182, 2183, 2184, 2185, 2186, 2187, 2188, 2189, 2190, 2191, 2192, 2193, 2194, 2195, 2196, 2197, 2198, 2199, 2200, 2201, 2202, 2203, 2204, 2205, 2206, 2207, 2208, 2209, 2210, 2211, 2212, 2213, 2214, 2215, 2216, 2217, 2218, 2219, 2220, 2221, 2222, 2223, 2224, 2225, 2226, 2227, 2228, 2229, 2230, 2231, 2232, 2233, 2234, 2235, 2236, 2237, 2238, 2239, 2240, 2241, 2242, 2243, 2244, 2245, 2246, 2247, 2248, 2249, 2250, 2251, 2252, 2253, 2254, 2255, 2256, 2257, 2258, 2259, 2260, 2261, 2262, 2263, 2264, 2265, 2266, 2267, 2268, 2269, 2270, 2271, 2272, 2273, 2274, 2275, 2276, 2277, 2278, 2279, 2280, 2281, 2282, 2283, 2284, 2285, 2286, 2287, 2288, 2289, 2290, 2291, 2292, 2293, 2294, 2295, 2296, 2297, 2298, 2299, 2300, 2301, 2302, 2303, 2304, 2305, 2306, 2307, 2308, 2309, 2310, 2311, 2312, 2313, 2314, 2315, 2316, 2317, 2318, 2319, 2320, 2321, 2322, 2323, 2324, 2325, 2326, 2327, 2328, 2329, 2330, 2331, 2332, 2333, 2334, 2335, 2336, 2337, 2338, 2339, 2340, 2341, 2342, 2343, 2344, 2345, 2346, 2347, 2348, 2349, 2350, 2351, 2352, 2353, 2354, 2355, 2356, 2357, 2358, 2359, 2360, 2361, 2362, 2363, 2364, 2365, 2366, 2367, 2368, 2369, 2370, 2371, 2372, 2373, 2374, 2375, 2376, 2377, 2378, 2379, 2380, 2381, 2382, 2383, 2384, 2385, 2386, 2387, 2388, 2389, 2390, 2391, 2392, 2393, 2394, 2395, 2396, 2397, 2398, 2399, 2400, 2401, 2402, 2403, 2404, 2405, 2406, 2407, 2408, 2409, 2410, 2411, 2412, 2413, 2414, 2415, 2416, 2417, 2418, 2419, 2420, 2421, 2422, 2423, 2424, 2425, 2426, 2427, 2428, 2429, 2430, 2431, 2432, 2433, 2434, 2435, 2436, 2437, 2438, 2439, 2440, 2441, 2442, 2443, 2444, 2445, 2446, 2447, 2448, 2449, 2450, 2451, 2452, 2453, 2454, 2455, 2456, 2457, 2458, 2459, 2460, 2461, 2462, 2463, 2464, 2465, 2466, 2467, 2468, 2469, 2470, 2471, 2472, 2473, 2474, 2475, 2476, 2477, 2478, 2479, 2480, 2481, 2482, 2483, 2484, 2485, 2486, 2487, 2488, 2489, 2490, 2491, 2492, 2493, 2494, 2495, 2496, 2497, 2498, 2499, 2500, 2501, 2502, 2503, 2504, 2505, 2506, 2507, 2508, 2509, 2510, 2511, 2512, 2513, 2514, 2515, 2516, 2517, 2518, 2519, 2520, 2521, 2522, 2523, 2524, 2525, 2526, 2527, 2528, 2529, 2530, 2531, 2532, 2533, 2534, 2535, 2536, 2537, 2538, 2539, 2540, 2541, 2542, 2543, 2544, 2545, 2546, 2547, 2548, 2549, 2550, 2551, 2552, 2553, 2554, 2555, 2556, 2557, 2558, 2559, 2560, 2561, 2562, 2563, 2564, 2565, 2566, 2567, 2568, 2569, 2570, 2571, 2572, 2573, 2574, 2575, 2576, 2577, 2578, 2579, 2580, 2581, 2582, 2583, 2584, 2585, 2586, 2587, 2588, 2589, 2590, 2591, 2592, 2593, 2594, 2595, 2596, 2597, 2598, 2599, 2600, 2601, 2602, 2603, 2604, 2605, 2606, 2607, 2608, 2609, 2610, 2611, 2612, 2613, 2614, 2615, 2616, 2617, 2618, 2619, 2620, 2621, 2622, 2623, 2624, 2625, 2626, 2627, 2628, 2629, 2630, 2631, 2632, 2633, 2634, 2635, 2636, 2637, 2638, 2639, 2640, 2641, 2642, 2643, 2644, 2645, 2646, 2647, 2648, 2649, 2650, 2651, 2652, 2653, 2654, 2655, 2656, 2657, 2658, 2659, 2660, 2661, 2662, 2663, 2664, 2665, 2666, 2667, 2668, 2669, 2670, 2671, 2672, 2673, 2674, 2675, 2676, 2677, 2678, 2679, 2680, 2681, 2682, 2683, 2684, 2685, 2686, 2687, 2688, 2689, 2690, 2691, 2692, 2693, 2694, 2695, 2696, 2697, 2698, 2699, 2700, 2701, 2702, 2703, 2704, 2705, 2706, 2707, 2708, 2709, 2710, 2711, 2712, 2713, 2714, 2715, 2716, 2717, 2718, 2719, 2720, 2721, 2722, 2723, 2724, 2725, 2726, 2727, 2728, 2729, 2730, 2731, 2732, 2733, 2734, 2735, 2736, 2737, 2738, 2739, 2740, 2741, 2742, 2743, 2744, 2745, 2746, 2747, 2748, 2749, 2750, 2751, 2752, 2753, 2754, 2755, 2756, 2757, 2758, 2759, 2760, 2761, 2762, 2763, 2764, 2765, 2766, 2767, 2768, 2769, 2770, 2771, 2772, 2773, 2774, 2775, 2776, 2777, 2778, 2779, 2780, 2781, 2782, 2783, 2784, 2785, 2786, 2787, 2788, 2789, 2790, 2791, 2792, 2793, 2794, 2795, 2796, 2797, 2798, 2799, 2800, 2801, 2802, 2803, 2804, 2805, 2806, 2807, 2808, 2809, 2810, 2811, 2812, 2813, 2814, 2815, 2816, 2817, 2818, 2819, 2820, 2821, 2822, 2823, 2824, 2825, 2826, 2827, 2828, 2829, 2830, 2831, 2832, 2833, 2834, 2835, 2836, 2837, 2838, 2839, 2840, 2841, 2842, 2843, 2844, 2845, 2846, 2847, 2848, 2849, 2850, 2851, 2852, 2853, 2854, 2855, 2856, 2857, 2858, 2859, 2860, 2861, 2862, 2863, 2864, 2865, 2866, 2867, 2868, 2869, 2870, 2871, 2872, 2873, 2874, 2875, 2876, 2877, 2878, 2879, 2880, 2881, 2882, 2883, 2884, 2885, 2886, 2887, 2888, 2889, 2890, 2891, 2892, 2893, 2894, 2895, 2896, 2897, 2898, 2899, 2900, 2901, 2902, 2903, 2904, 2905, 2906, 2907, 2908, 2909, 2910, 2911, 2912, 2913, 2914, 2915, 2916, 2917, 2918, 2919, 2920, 2921, 2922, 2923, 2924, 2925, 2926, 2927, 2928, 2929, 2930, 2931, 2932, 2933, 2934, 2935, 2936, 2937, 2938, 2939, 2940, 2941, 2942, 2943, 2944, 2945, 2946, 2947, 2948, 2949, 2950, 2951, 2952, 2953, 2954, 2955, 2956, 2957, 2958, 2959, 2960, 2961, 2962, 2963, 2964, 2965, 2966, 2967, 2968, 2969, 2970, 2971, 2972, 2973, 2974, 2975, 2976, 2977, 2978, 2979, 2980, 2981, 2982, 2983, 2984, 2985, 2986, 2987, 2988, 2989, 2990, 2991, 2992, 2993, 2994, 2995, 2996, 2997, 2998, 2999, 3000, 3001, 3002, 3003, 3004, 3005, 3006, 3007, 3008, 3009, 3010, 3011, 3012, 3013, 3014, 3015, 3016, 3017, 3018, 3019, 3020, 3021, 3022, 3023, 3024, 3025, 3026, 3027, 3028, 3029, 3030, 3031, 3032, 3033, 3034, 3035, 3036, 3037, 3038, 3039, 3040, 3041, 3042, 3043, 3044, 3045, 3046, 3047, 3048, 3049, 3050, 3051, 3052, 3053, 3054, 3055, 3056, 3057, 3058, 3059, 3060, 3061, 3062, 3063, 3064, 3065, 3066, 3067, 3068, 3069, 3070, 3071, 3072, 3073, 3074, 3075, 3076, 3077, 3078, 3079, 3080, 3081, 3082, 3083, 3084, 3085, 3086, 3087, 3088, 3089, 3090, 3091, 3092, 3093, 3094, 3095, 3096, 3097, 3098, 3099, 3100, 3101, 3102, 3103, 3104, 3105, 3106, 3107, 3108, 3109, 3110, 3111, 3112, 3113, 3114, 3115, 3116, 3117, 3118, 3119, 3120, 3121, 3122, 3123, 3124, 3125, 3126, 3127, 3128, 3129, 3130, 3131, 3132, 3133, 3134, 3135, 3136, 3137, 3138, 3139, 3140, 3141, 3142, 3143, 3144, 3145, 3146, 3147, 3148, 3149, 3150, 3151, 3152, 3153, 3154, 3155, 3156, 3157, 3158, 3159, 3160, 3161, 3162, 3163, 3164, 3165, 3166, 3167, 3168, 3169, 3170, 3171, 3172, 3173, 3174, 3175, 3176, 3177, 3178, 3179, 3180, 3181, 3182, 3183, 3184, 3185, 3186, 3187, 3188, 3189, 3190, 3191, 3192, 3193, 3194, 3195, 3196, 3197, 3198, 3199, 3200, 3201, 3202, 3203, 3204, 3205, 3206, 3207, 3208, 3209, 3210, 3211, 3212, 3213, 3214, 3215, 3216, 3217, 3218, 3219, 3220, 3221, 3222, 3223, 3224, 3225, 3226, 3227, 3228, 3229, 3230, 3231, 3232, 3233, 3234, 3235, 3236, 3237, 3238, 3239, 3240, 3241, 3242, 3243, 3244, 3245, 3246, 3247, 3248, 3249, 3250, 3251, 3252, 3253, 3254, 3255, 3256, 3257, 3258, 3259, 3260, 3261, 3262, 3263, 3264, 3265, 3266, 3267, 3268, 3269, 3270, 3271, 3272, 3273, 3274, 3275, 3276, 3277, 3278, 3279, 3280, 3281, 3282, 3283, 3284, 3285, 3286, 3287, 3288, 3289, 3290, 3291, 3292, 3293, 3294, 3295, 3296, 3297, 3298, 3299, 3300, 3301, 3302, 3303, 3304, 3305, 3306, 3307, 3308, 3309, 3310, 3311, 3312, 3313, 3314, 3315, 3316, 3317, 3318, 3319, 3320, 3321, 3322, 3323, 3324, 3325, 3326, 3327, 3328, 3329, 3330, 3331, 3332, 3333, 3334, 3335, 3336, 3337, 3338, 3339, 3340, 3341, 3342, 3343, 3344, 3345, 3346, 3347, 3348, 3349, 3350, 3351, 3352, 3353, 3354, 3355, 3356, 3357, 3358, 3359, 3360, 3361, 3362, 3363, 3364, 3365, 3366, 3367, 3368, 3369, 3370, 3371, 3372, 3373, 3374, 3375, 3376, 3377, 3378, 3379, 3380, 3381, 3382, 3383, 3384, 3385, 3386, 3387, 3388, 3389, 3390, 3391, 3392, 3393, 3394, 3395, 3396, 3397, 3398, 3399, 3400, 3401, 3402, 3403, 3404, 3405, 3406, 3407, 3408, 3409, 3410, 3411, 3412, 3413, 3414, 3415, 3416, 3417, 3418, 3419, 3420, 3421, 3422, 3423, 3424, 3425, 3426, 3427, 3428, 3429, 3430, 3431, 3432, 3433, 3434, 3435, 3436, 3437, 3438, 3439, 3440, 3441, 3442, 3443, 3444, 3445, 3446, 3447, 3448, 3449, 3450, 3451, 3452, 3453, 3454, 3455, 3456, 3457, 3458, 3459, 3460, 3461, 3462, 3463, 3464, 3465, 3466, 3467, 3468, 3469, 3470, 3471, 3472, 3473, 3474, 3475, 3476, 3477, 3478, 3479, 3480, 3481, 3482, 3483, 3484, 3485, 3486, 3487, 3488, 3489, 3490, 3491, 3492, 3493, 3494, 3495, 3496, 3497, 3498, 3499, 3500, 3501, 3502, 3503, 3504, 3505, 3506, 3507, 3508, 3509, 3510, 3511, 3512, 3513, 3514, 3515, 3516, 3517, 3518, 3519, 3520, 3521, 3522, 3523, 3524, 3525, 3526, 3527, 3528, 3529, 3530, 3531, 3532, 3533, 3534, 3535, 3536, 3537, 3538, 3539, 3540, 3541, 3542, 3543, 3544, 3545, 3546, 3547, 3548, 3549, 3550, 3551, 3552, 3553, 3554, 3555, 3556, 3557, 3558, 3559, 3560, 3561, 3562, 3563, 3564, 3565, 3566, 3567, 3568, 3569, 3570, 3571, 3572, 3573, 3574, 3575, 3576, 3577, 3578, 3579, 3580, 3581, 3582, 3583, 3584, 3585, 3586, 3587, 3588, 3589, 3590, 3591, 3592, 3593, 3594, 3595, 3596, 3597, 3598, 3599, 3600, 3601, 3602, 3603, 3604, 3605, 3606, 3607, 3608, 3609, 3610, 3611, 3612, 3613, 3614, 3615, 3616, 3617, 3618, 3619, 3620, 3621, 3622, 3623, 3624, 3625, 3626, 3627, 3628, 3629, 3630, 3631, 3632, 3633, 3634, 3635, 3636, 3637, 3638, 3639, 3640, 3641, 3642, 3643, 3644, 3645, 3646, 3647, 3648, 3649, 3650, 3651, 3652, 3653, 3654, 3655, 3656, 3657, 3658, 3659, 3660, 3661, 3662, 3663, 3664, 3665, 3666, 3667, 3668, 3669, 3670, 3671, 3672, 3673, 3674, 3675, 3676, 3677, 3678, 3679, 3680, 3681, 3682, 3683, 3684, 3685, 3686, 3687, 3688, 3689, 3690, 3691, 3692, 3693, 3694, 3695, 3696, 3697, 3698, 3699, 3700, 3701, 3702, 3703, 3704, 3705, 3706, 3707, 3708, 3709, 3710, 3711, 3712, 3713, 3714, 3715, 3716, 3717, 3718, 3719, 3720, 3721, 3722, 3723, 3724, 3725, 3726, 3727, 3728, 3729, 3730, 3731, 3732, 3733, 3734, 3735, 3736, 3737, 3738, 3739, 3740, 3741, 3742, 3743, 3744, 3745, 3746, 3747, 3748, 3749, 3750, 3751, 3752, 3753, 3754, 3755, 3756, 3757, 3758, 3759, 3760, 3761, 3762, 3763, 3764, 3765, 3766, 3767, 3768, 3769, 3770, 3771, 3772, 3773, 3774, 3775, 3776, 3777, 3778, 3779, 3780, 3781, 3782, 3783, 3784, 3785, 3786, 3787, 3788, 3789, 3790, 3791, 3792, 3793, 3794, 3795, 3796, 3797, 3798, 3799, 3800, 3801, 3802, 3803, 3804, 3805, 3806, 3807, 3808, 3809, 3810, 3811, 3812, 3813, 3814, 3815, 3816, 3817, 3818, 3819, 3820, 3821, 3822, 3823, 3824, 3825, 3826, 3827, 3828, 3829, 3830, 3831, 3832, 3833, 3834, 3835, 3836, 3837, 3838, 3839, 3840, 3841, 3842, 3843, 3844, 3845, 3846, 3847, 3848, 3849, 3850, 3851, 3852, 3853, 3854, 3855, 3856, 3857, 3858, 3859, 3860, 3861, 3862, 3863, 3864, 3865, 3866, 3867, 3868, 3869, 3870, 3871, 3872, 3873, 3874, 3875, 3876, 3877, 3878, 3879, 3880, 3881, 3882, 3883, 3884, 3885, 3886, 3887, 3888, 3889, 3890, 3891, 3892, 3893, 3894, 3895, 3896, 3897, 3898, 3899, 3900, 3901, 3902, 3903, 3904, 3905, 3906, 3907, 3908, 3909, 3910, 3911, 3912, 3913, 3914, 3915, 3916, 3917, 3918, 3919, 3920, 3921, 3922, 3923, 3924, 3925, 3926, 3927, 3928, 3929, 3930, 3931, 3932, 3933, 3934, 3935, 3936, 3937, 3938, 3939, 3940, 3941, 3942, 3943, 3944, 3945, 3946, 3947, 3948, 3949, 3950, 3951, 3952, 3953, 3954, 3955, 3956, 3957, 3958, 3959, 3960, 3961, 3962, 3963, 3964, 3965, 3966, 3967, 3968, 3969, 3970, 3971, 3972, 3973, 3974, 3975, 3976, 3977, 3978, 3979, 3980, 3981, 3982, 3983, 3984, 3985, 3986, 3987, 3988, 3989, 3990, 3991, 3992, 3993, 3994, 3995, 3996, 3997, 3998, 3999, 4000, 4001, 4002, 4003, 4004, 4005, 4006, 4007, 4008, 4009, 4010, 4011, 4012, 4013, 4014, 4015, 4016, 4017, 4018, 4019, 4020, 4021, 4022, 4023, 4024, 4025, 4026, 4027, 4028, 4029, 4030, 4031, 4032, 4033, 4034, 4035, 4036, 4037, 4038, 4039, 4040, 4041, 4042, 4043, 4044, 4045, 4046, 4047, 4048, 4049, 4050, 4051, 4052, 4053, 4054, 4055, 4056, 4057, 4058, 4059, 4060, 4061, 4062, 4063, 4064, 4065, 4066, 4067, 4068, 4069, 4070, 4071, 4072, 4073, 4074, 4075, 4076, 4077, 4078, 4079, 4080, 4081, 4082, 4083, 4084, 4085, 4086, 4087, 4088, 4089, 4090, 4091, 4092, 4093, 4094, 4095, 4096, 4097, 4098, 4099, 4100, 4101, 4102, 4103, 4104, 4105, 4106, 4107, 4108, 4109, 4110, 4111, 4112, 4113, 4114, 4115, 4116, 4117, 4118, 4119, 4120, 4121, 4122, 4123, 4124, 4125, 4126, 4127, 4128, 4129, 4130, 4131, 4132, 4133, 4134, 4135, 4136, 4137, 4138, 4139, 4140, 4141, 4142, 4143, 4144, 4145, 4146, 4147, 4148, 4149, 4150, 4151, 4152, 4153, 4154, 4155, 4156, 4157, 4158, 4159, 4160, 4161, 4162, 4163, 4164, 4165, 4166, 4167, 4168, 4169, 4170, 4171, 4172, 4173, 4174, 4175, 4176, 4177, 4178, 4179, 4180, 4181, 4182, 4183, 4184, 4185, 4186, 4187, 4188, 4189, 4190, 4191, 4192, 4193, 4194, 4195, 4196, 4197, 4198, 4199, 4200, 4201, 4202, 4203, 4204, 4205, 4206, 4207, 4208, 4209, 4210, 4211, 4212, 4213, 4214, 4215, 4216, 4217, 4218, 4219, 4220, 4221, 4222, 4223, 4224, 4225, 4226, 4227, 4228, 4229, 4230, 4231, 4232, 4233, 4234, 4235, 4236, 4237, 4238, 4239, 4240, 4241, 4242, 4243, 4244, 4245, 4246, 4247, 4248, 4249, 4250, 4251, 4252, 4253, 4254, 4255, 4256, 4257, 4258, 4259, 4260, 4261, 4262, 4263, 4264, 4265, 4266, 4267, 4268, 4269, 4270, 4271, 4272, 4273, 4274, 4275, 4276, 4277, 4278, 4279, 4280, 4281, 4282, 4283, 4284, 4285, 4286, 4287, 4288, 4289, 4290, 4291, 4292, 4293, 4294, 4295, 4296, 4297, 4298, 4299, 4300, 4301, 4302, 4303, 4304, 4305, 4306, 4307, 4308, 4309, 4310, 4311, 4312, 4313, 4314, 4315, 4316, 4317, 4318, 4319, 4320, 4321, 4322, 4323, 4324, 4325, 4326, 4327, 4328, 4329, 4330, 4331, 4332, 4333, 4334, 4335, 4336, 4337, 4338, 4339, 4340, 4341, 4342, 4343, 4344, 4345, 4346, 4347, 4348, 4349, 4350, 4351, 4352, 4353, 4354, 4355, 4356, 4357, 4358, 4359, 4360, 4361, 4362, 4363, 4364, 4365, 4366, 4367, 4368, 4369, 4370, 4371, 4372, 4373, 4374, 4375, 4376, 4377, 4378, 4379, 4380, 4381, 4382, 4383, 4384, 4385, 4386, 4387, 4388, 4389, 4390, 4391, 4392, 4393, 4394, 4395, 4396, 4397, 4398, 4399, 4400, 4401, 4402, 4403, 4404, 4405, 4406, 4407, 4408, 4409, 4410, 4411, 4412, 4413, 4414, 4415, 4416, 4417, 4418, 4419, 4420, 4421, 4422, 4423, 4424, 4425, 4426, 4427, 4428, 4429, 4430, 4431, 4432, 4433, 4434, 4435, 4436, 4437, 4438, 4439, 4440, 4441, 4442, 4443, 4444, 4445, 4446, 4447, 4448, 4449, 4450, 4451, 4452, 4453, 4454, 4455, 4456, 4457, 4458, 4459, 4460, 4461, 4462, 4463, 4464, 4465, 4466, 4467, 4468, 4469, 4470, 4471, 4472, 4473, 4474, 4475, 4476, 4477, 4478, 4479, 4480, 4481, 4482, 4483, 4484, 4485, 4486, 4487, 4488, 4489, 4490, 4491, 4492, 4493, 4494, 4495, 4496, 4497, 4498, 4499, 4500, 4501, 4502, 4503, 4504, 4505, 4506, 4507, 4508, 4509, 4510, 4511, 4512, 4513, 4514, 4515, 4516, 4517, 4518, 4519, 4520, 4521, 4522, 4523, 4524, 4525, 4526, 4527, 4528, 4529, 4530, 4531, 4532, 4533, 4534, 4535, 4536, 4537, 4538, 4539, 4540, 4541, 4542, 4543, 4544, 4545, 4546, 4547, 4548, 4549, 4550, 4551, 4552, 4553, 4554, 4555, 4556, 4557, 4558, 4559, 4560, 4561, 4562, 4563, 4564, 4565, 4566, 4567, 4568, 4569, 4570, 4571, 4572, 4573, 4574, 4575, 4576, 4577, 4578, 4579, 4580, 4581, 4582, 4583, 4584, 4585, 4586, 4587, 4588, 4589, 4590, 4591, 4592, 4593, 4594, 4595, 4596, 4597, 4598, 4599, 4600, 4601, 4602, 4603, 4604, 4605, 4606, 4607, 4608, 4609, 4610, 4611, 4612, 4613, 4614, 4615, 4616, 4617, 4618, 4619, 4620, 4621, 4622, 4623, 4624, 4625, 4626, 4627, 4628, 4629, 4630, 4631, 4632, 4633, 4634, 4635, 4636, 4637, 4638, 4639, 4640, 4641, 4642, 4643, 4644, 4645, 4646, 4647, 4648, 4649, 4650, 4651, 4652, 4653, 4654, 4655, 4656, 4657, 4658, 4659, 4660, 4661, 4662, 4663, 4664, 4665, 4666, 4667, 4668, 4669, 4670, 4671, 4672, 4673, 4674, 4675, 4676, 4677, 4678, 4679, 4680, 4681, 4682, 4683, 4684, 4685, 4686, 4687, 4688, 4689, 4690, 4691, 4692, 4693, 4694, 4695, 4696, 4697, 4698, 4699, 4700, 4701, 4702, 4703, 4704, 4705, 4706, 4707, 4708, 4709, 4710, 4711, 4712, 4713, 4714, 4715, 4716, 4717, 4718, 4719, 4720, 4721, 4722, 4723, 4724, 4725, 4726, 4727, 4728, 4729, 4730, 4731, 4732, 4733, 4734, 4735, 4736, 4737, 4738, 4739, 4740, 4741, 4742, 4743, 4744, 4745, 4746, 4747, 4748, 4749, 4750, 4751, 4752, 4753, 4754, 4755, 4756, 4757, 4758, 4759, 4760, 4761, 4762, 4763, 4764, 4765, 4766, 4767, 4768, 4769, 4770, 4771, 4772, 4773, 4774, 4775, 4776, 4777, 4778, 4779, 4780, 4781, 4782, 4783, 4784, 4785, 4786, 4787, 4788, 4789, 4790, 4791, 4792, 4793, 4794, 4795, 4796, 4797, 4798, 4799, 4800, 4801, 4802, 4803, 4804, 4805, 4806, 4807, 4808, 4809, 4810, 4811, 4812, 4813, 4814, 4815, 4816, 4817, 4818, 4819, 4820, 4821, 4822, 4823, 4824, 4825, 4826, 4827, 4828, 4829, 4830, 4831, 4832, 4833, 4834, 4835, 4836, 4837, 4838, 4839, 4840, 4841, 4842, 4843, 4844, 4845, 4846, 4847, 4848, 4849, 4850, 4851, 4852, 4853, 4854, 4855, 4856, 4857, 4858, 4859, 4860, 4861, 4862, 4863, 4864, 4865, 4866, 4867, 4868, 4869, 4870, 4871, 4872, 4873, 4874, 4875, 4876, 4877, 4878, 4879, 4880, 4881, 4882, 4883, 4884, 4885, 4886, 4887, 4888, 4889, 4890, 4891, 4892, 4893, 4894, 4895, 4896, 4897, 4898, 4899, 4900, 4901, 4902, 4903, 4904, 4905, 4906, 4907, 4908, 4909, 4910, 4911, 4912, 4913, 4914, 4915, 4916, 4917, 4918, 4919, 4920, 4921, 4922, 4923, 4924, 4925, 4926, 4927, 4928, 4929, 4930, 4931, 4932, 4933, 4934, 4935, 4936, 4937, 4938, 4939, 4940, 4941, 4942, 4943, 4944, 4945, 4946, 4947, 4948, 4949, 4950, 4951, 4952, 4953, 4954, 4955, 4956, 4957, 4958, 4959, 4960, 4961, 4962, 4963, 4964, 4965, 4966, 4967, 4968, 4969, 4970, 4971, 4972, 4973, 4974, 4975, 4976, 4977, 4978, 4979, 4980, 4981, 4982, 4983, 4984, 4985, 4986, 4987, 4988, 4989, 4990, 4991, 4992, 4993, 4994, 4995, 4996, 4997, 4998, 4999 and 5000 ppm.

In some embodiments, the emulsions, or the micelles, or the liposomes, or the hydrogels, or the dry powders, or the oil solutions of the invention comprise a halocarbon compound (preferably bromoform) wherein the concentration of the halocarbon compound (preferably bromoform) is stable for a period of time falling within the range of about 1 day to about 600 days.

In some embodiments, the emulsions, or the micelles, or the liposomes, or the hydrogels, or the dry powders, or the oil solutions of the invention comprise a halocarbon compound (preferably bromoform) wherein the concentration of the halocarbon compound (preferably bromoform) is stable for a period of time selected from the group consisting of; 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295, 296, 297, 298, 299, 300, 301, 302, 303, 304, 305, 306, 307, 308, 309, 310, 311, 312, 313, 314, 315, 316, 317, 318, 319, 320, 321, 322, 323, 324, 325, 326, 327, 328, 329, 330, 331, 332, 333, 334, 335, 336, 337, 338, 339, 340, 341, 342, 343, 344, 345, 346, 347, 348, 349, 350, 351, 352, 353, 354, 355, 356, 357, 358, 359, 360, 361, 362, 363, 364, 365, 366, 367, 368, 369, 370, 371, 372, 373, 374, 375, 376, 377, 378, 379, 380, 381, 382, 383, 384, 385, 386, 387, 388, 389, 390, 391, 392, 393, 394, 395, 396, 397, 398, 399, 400, 401, 402, 403, 404, 405, 406, 407, 408, 409, 410, 411, 412, 413, 414, 415, 416, 417, 418, 419, 420, 421, 422, 423, 424, 425, 426, 427, 428, 429, 430, 431, 432, 433, 434, 435, 436, 437, 438, 439, 440, 441, 442, 443, 444, 445, 446, 447, 448, 449, 450, 451, 452, 453, 454, 455, 456, 457, 458, 459, 460, 461, 462, 463, 464, 465, 466, 467, 468, 469, 470, 471, 472, 473, 474, 475, 476, 477, 478, 479, 480, 481, 482, 483, 484, 485, 486, 487, 488, 489, 490, 491, 492, 493, 494, 495, 496, 497, 498, 499, 500, 501, 502, 503, 504, 505, 506, 507, 508, 509, 510, 511, 512, 513, 514, 515, 516, 517, 518, 519, 520, 521, 522, 523, 524, 525, 526, 527, 528, 529, 530, 531, 532, 533, 534, 535, 536, 537, 538, 539, 540, 541, 542, 543, 544, 545, 546, 547, 548, 549, 550, 551, 552, 553, 554, 555, 556, 557, 558, 559, 560, 561, 562, 563, 564, 565, 566, 567, 568, 569, 570, 571, 572, 573, 574, 575, 576, 577, 578, 579, 580, 581, 582, 583, 584, 585, 586, 587, 588, 589, 590, 591, 592, 593, 594, 595, 596, 597, 598, 599, and 600 days.

In a preferred embodiment, the concentration of the halocarbon compound within the compositions of the invention remains stable for a period of time of at least about 2, 5, 7, 9, 11, 15, 30, 60, 90, 120, 150, 180, 210, 240, 270, 300, 330, 360, 390, 420, 450, 480, 510, 540, 570 or 600 days, preferably as determined by ICP-AES.

In one embodiment, the present invention relates to the compositions of the invention, when used to inhibit methanogenesis in a ruminant.

In one embodiment, the present invention relates to the use of the compositions of the invention, to inhibit methanogenesis in a ruminant.

In one embodiment, the disclosure of the invention herein provides a method of administering a composition comprising a stabilised halocarbon compound to a ruminant, wherein the composition reduces and/or inhibits methanogenesis in the ruminant.

In a particularly preferred embodiment, the disclosure of the invention herein provides a method of administering a composition comprising stabilised bromoform to a ruminant, wherein the composition reduces and/or inhibits methanogenesis in the ruminant.

In a particularly preferred embodiment, the disclosure of the invention herein provides a method of administering a composition comprising stabilised bromoform to a ruminant, wherein the composition reduces and/or inhibits methanogenesis in the ruminant without significantly affecting total Volatile Fatty Acid (VFA) production in the rumen; and/or without significantly affecting total gas production in the rumen; and/or without significantly affecting Acetate:Propionate (A:P) levels in the rumen; and/or whilst advantageously reducing NH3 levels in the rumen.

### Definitions

Throughout this specification, unless the context requires otherwise, the word “comprise” or variations such as “comprises” or “comprising”, will be understood to imply the inclusion of a stated integer or group of integers but not the exclusion of any other integer or group of integers.

As used herein, the term “halocarbon compound” includes any organic molecule wherein one or more hydrogen atom(s) has been replaced by one or more halogen atom(s) independently selected in each instance of replacement from the group consisting of fluorine, chlorine, bromine or iodine. The term “halocarbon compound” also includes halogen-containing reaction products of halocarbon compounds. Preferred examples of such halocarbon compounds include haloalkanes, haloalkenes, haloaromatics, halogenated carboxylic acids and their corresponding esters, haloalkyl alcohols, haloalkyl ethers, haloalkyl amines and salts thereof, haloalkyl thioethers and haloalkyl amides, including mixtures of any of the aforesaid halocarbon compounds, and/or metabolites of any of the aforesaid halocarbon compounds, and/or halogen-containing reaction products of any of the aforesaid halocarbon compounds. It is to be understood that the halocarbon compounds to be used in the formulations of the invention are synthetically derived and/or substantially pure halocarbon compounds, that are not derived as extracts or otherwise from seaweed sources such as red marine macroalgae, or recombinant organisms such as yeasts. It is further to be understood that the halocarbon compounds to be used in the formulations of the invention are not selected from the group consisting of chloroform, or carbon tetrachloride, or methylene chloride, or hexachloroethane, or bromochloromethane, or bromochloroethane, or bromochloropropane, or halothane, or 2-bromoethane sulphonate, or 2-bromo-2-chloroethane sulphonate.

Depending on the particular formulation of the composition, preferred halocarbon compounds include bromoform, bromochloroacetic acid, dibromoacetic acid, chloral hydrate, iodopropane, and dibromochloromethane.

As used herein, the term “metabolite” includes any molecular species produced by in vivo transformation of the subject molecular entity to which it is applied, including chemical transformation such as hydrolysis, enzymatic transformation such as lysis or other any other enzymatic transformation, and microbial transformation due to microflora present in the in vivo environment of the subject, and may include molecular species in liquid form, or in solution, or in solid form, or in gaseous form, such as, but not limited to, methane, carbon dioxide or hydrogen sulfide.

As used herein, the term “stabilised” refers to a property of inhibiting the reactivity or inhibiting the decomposition, or inhibiting the volatility or inhibiting the toxicity or inhibiting the environmental pollutant activity of the halocarbon compound(s) within the compositions of the present invention.

As used herein, the term “stabilising agent” includes any component within the compositions of the present invention that exhibits a property of inhibiting the reactivity or inhibiting the decomposition, or inhibiting the volatility or inhibiting the toxicity or inhibiting the environmental pollutant activity of the halocarbon compound(s) within the compositions of the present invention. Preferred examples of such stabilising agents include proteins (including globular proteins such as those present in milk), peptides, oligopeptides, amino acids, carbohydrates, saccharides, disaccharides, oligosaccharides polysaccharides, xanthan gum, (xanthum gum), guar gum, celluloses, starches, lipids, fatty acids, waxes, oils, sterols, fat-soluble vitamins (such as vitamins A, D, E, and K), monoglycerides, diglycerides, triglycerides, MCT, phospholipids, glycerolipids, sphingolipids, saccharolipids, polyketides, sterol lipids prenol lipids, emulsifying agents, glycerol, sorbitol, lecithins, glycerophospholipids, phosphatidylcholines, phosphatidylethanolamines, phosphatidylinositols, phosphatidylserines, phosphatidic acids, surfactants, food acids, diacetyl tartaric acid esters of mono- and diglycerides, detergents, encapsulating agents, alkyl celluloses, ethyl celluloses, polyvinyl alcohols, gelatins, carageenans, hydrogels, alginates alginate salts, edible polymers, biocompatible polymers, fresh milk, whole milk powder, or combinations of any of the aforementioned.

As used herein, the term “encapsulated” and variations of the term such as “encapsulation” and “encapsulating” includes microencapsulation or nanoencapsulation, and refers to a process in which tiny particles or droplets are surrounded by a coating to give small capsule. The coating material is referred to as an “encapsulating agent”. In its simplest and idealised form, a microcapsule is a small sphere comprising a near-uniform wall enclosing some material. In reality, microcapsules are rarely spherical and may assume any three-dimensional shape. The enclosed material in the microcapsule is referred to as the core, internal phase, or fill, whereas the wall is sometimes called a shell, coating, or membrane. Some materials like lipids and polymers, such as alginate, may be used as a mixture to trap the material of interest inside. Most microcapsules have pores with diameters between a few nanometers and a few micrometers. The coating materials generally used for coating include alkyl celluloses, polyvinyl alcohols, gelatins, alginate salts, and hydrogels. Many microcapsules however bear little resemblance to simple spheres. The core may be a crystal, a jagged adsorbent particle, or a liquid, and the shell can be facilitated via formation of an emulsion, a hydrogel, a micelle, a liposome, a Pickering emulsion, a suspension of solids, or a suspension of smaller microcapsules. The microcapsule even may have multiple walls. Encapsulation may be achieved by a number of processes including but not limited to; pan coating wherein particles are tumbled in a pan or other device; centrifugal extrusion wherein liquids are encapsulated by a rotating head containing concentric nozzles providing a jet of core liquid which is surrounded by a sheath of wall solution or melt; vibrational nozzle techniques wherein core-shell encapsulation or microgranulation (matrix-encapsulation) can be achieved via a laminar flow through a nozzle and an additional vibration of the nozzle or the liquid; spray-drying which serves as a microencapsulation technique when an active material is dissolved or suspended in a melt or polymer solution or emulsion and becomes trapped in the dried particle; physicochemical methods, including; ionotropic gelation wherein units of uric acid in the chains of alginate polymers crosslink with multivalent cations formed from calcium, zinc, iron or aluminium; coacervation-phase separation wherein continuous agitation is performed during formation of a three-phase system consisting of a liquid manufacturing vehicle phase, a core material phase and a coating material phase, and the coating is deposited via a process wherein core material is dispersed in the coating polymer solution, coating polymer material is coated around core, and deposition of liquid polymer coating around the core by polymer adsorbed at the interface formed between core material and vehicle phase occurs, followed by rigidization of the coating, wherein coating material immiscible in the vehicle phase and is made rigid via thermal, cross-linking, or dissolution techniques; and chemical methods, including; interfacial polycondensation, whereby the two reactants in a polycondensation meet at an interface and react rapidly, based on the classical Schotten-Baumann reaction between an acid chloride and a compound containing an active hydrogen atom, such as an amine or alcohol, or biocompatible polymers including polyesters, polyurea, polyurethane, forming thin flexible walls rapidly at the interface; interfacial cross-linking, involving a similar process to interfacial polycondensation but replacing bifunctional monomers containing active hydrogen atoms with biosourced polymers such as proteins, and whereby when the reaction is performed at the interface of an emulsion, the acid chloride reacts with the various functional groups of the biosourced polymer, leading to the formation of a membrane; in-situ polymerisation wherein the direct polymerization of a single monomer is carried out on the core particle surface; and matrix polymerisation, whereby a core material is imbedded in a polymeric matrix during formation of the particles, such as occurs during spray-drying, in which the particle is formed by evaporation of the solvent from the matrix material, or via the solidification of the matrix due to a chemical change.

As used herein, the term “liposome” is to be understood to mean a small artificial vesicle, spherical in shape, having at least one lipid bilayer. Since liposomes are artificial, they will be understood to exclude any cellular structures of biological origin, such as red marine macroalgae cells, and yeast cells, and/or any vesicles derived from biological organisms.

The term “active agent” may mean one active agent, or may encompass two or more active agents. Without limitations the active agent can be selected from the group consisting of small organic or inorganic molecules, saccharines, oligosaccharides, polysaccharides, peptides, proteins, silk fibroin, peptide analogs and derivatives, peptidomimetics, nucleic acids, nucleic acid analogs and derivatives, antibodies, antigen binding fragments of antibodies, lipids, extracts made from biological materials, naturally occurring or synthetic compositions, biologics, biosimilars and any combinations thereof. Active agents may include, without limitation, additional antimethanogenic agents such as but not limited to 3-nitrooxypropanol (3-NOP), anti-microbial agents; anti-inflammatory agents; stem cells; transcription factors; antiproliferative agents; anticancer agents; growth factors; hormones; steroids; enzyme or receptor modulators; hydroxyapatite; tricalcium phosphate; polyhedral oligomeric silsesquioxane (POSS); POSS derivatives; pyrithione salts; ketoconazole; salicylic acid; curcumin or a derivative of curcumin, curcuminoids; tetrahydro curcuminoids; titanium dioxide (TiCk); zinc oxide (ZnO); chloroxylenol; flavanoids; CoQIO; vitamin C; herbal extracts; alkaloids; 13-cis retinoic acid; 3,4-methylenedioxymethamphetamine; 5-fluorouracil; 6,8-dimercaptooctanoic acid (dihydrolipoic acid); abacavir; acebutolol; acetaminophen; acetaminosalol; acetazolamide; acetohydroxamic acid; acetylsalicylic acid; acitretin; aclovate; acrivastine; actiq; acyclovir; adapalene; adefovir dipivoxil; adenosine; Albaconazole; albuterol; alfuzosin; Allicin; allopurinol; alloxanthine; allylamines; almotriptan; alpha-hydroxy acids; alprazolam; alprenolol; aluminum acetate; aluminum chloride; aluminum chlorohydroxide; aluminum hydroxide; amantadine; amiloride; aminacrine; aminobenzoic acid (PABA); aminocaproic acid; aminoglycosides such as streptomycin, neomycin, kanamycin, paromycin, gentamicin, tobramycin, amikacin, netilmicin, spectinomycin, sisomicin, dibekalin and isepamicin; aminosalicylic acid; amiodarone; amitriptyline; amlodipine; amocarzine; amodiaquin; Amorolfin; amoxapine; amphetamine; amphotericin B; ampicillin; anagrelide; anastrozole; Anidulafungin; anthralin; antibacterial sulfonamides and antibacterial sulphanilamides, including para-aminobenzoic acid, sulfadiazine, sulfisoxazole, sulfamethoxazole and sulfathalidine; antifungal peptide and derivatives and analogs thereof; apomorphine; aprepitant; arbutin; aripiprazole; ascorbic acid; ascorbyl palmitate; atazanavir; atenolol; atomoxetine; atropine; azathioprine; azelaic acid; azelaic acid; azelastine; azithromycin; bacitracin; bacitracin; beanomicins; beclomefhasone dipropionate; bemegride; benazepril; bendroflumethiazide; benzocaine; Benzoic acid with a keratolytic agent; benzonatate; benzophenone; benztropine; bepridil; beta-hydroxy acids; beta-lactams including penicillin, cephalosporin, and carbapenems such as carbapenem, imipenem, and meropenem; betamethasone dipropionate; betamethasone valerate; brimonidine; brompheniramine; bupivacaine; buprenorphine; bupropion; burimamide; butenafine; Butenafine; butoconazole; Butoconazole; cabergoline; caffeic acid; caffeine; calcipotriene; camphor; Cancidas; candesartan cilexetil; capsaicin; carbamazepine; Caspofungin; cefditoren pivoxil; cefepime; cefpodoxime proxetil; celecoxib; cetirizine; cevimeline; chitosan; chlordiazepoxide; chlorhexidine; chloroquine; chlorothiazide; chloroxylenol; chlorpheniramine; chlorpromazine; chlorpropamide; ciclopirox; Ciclopirox (ciclopirox olamine); cilostazol; cimetidine; cinacalcet; ciprofloxacin; citalopram; citric acid; Citronella oil; cladribine; clarithromycin; clemastine; clindamycin; clioquinol; clobetasol propionate; clomiphene; clonidine; clopidogrel; Clortrimazole; clotrimazole; Clotrimazole; clozapine; cocaine; Coconut oil; codeine; colistin; colymycin; cromolyn; crotamiton; Crystal violet; cyclizine; cyclobenzaprine; cycloserine; cytarabine; dacarbazine; dalfopristin; dapsone; daptomycin; daunorubicin; deferoxamine: dehydroepiandrosterone; delavirdine; desipramine; desloratadine; desmopressin; desoximetasone; dexamethasone; dexmedetomidine; dexmethylphenidate; dexrazoxane; dextroamphetamine; diazepam; dicyclomine; didanosine; dihydrocodeine; dihydromorphine; diltiazem; diphenhydramine; diphenoxylate; dipyridamole; disopyramide; dobutamine; dofetilide; dolasetron; donepezil; dopa esters; dopamine; dopamnide; dorzolamide; doxepin; doxorubicin; doxycycline; doxylamine; doxypin; duloxetine; dyclonine; echinocandins; econazole; Econazole; eflormthine; eletriptan; emtricitabine; enalapril; ephedrine; epinephrine; epinine; epirubicin; eptifibatide; ergotamine; erythromycin; escitalopram; esmolol; esomeprazole; estazolam; estradiol; ethacrynic acid; ethinyl estradiol; etidocaine; etomidate; famciclovir; famotidine; felodipine; fentanyl; Fenticonazole; ferulic acid; fexofenadine; flecainide; fluconazole; Fluconazole; flucytosiine; Flucytosine or 5-fluorocytosine; fluocinolone acetonide; fluocinonide; fluoxetine; fluphenazine; flurazepam; fluvoxamine; formoterol; furosemide; galactarolactone; galactonic acid; galactonolactone; galactose; galantamine; gatifloxacin; gefitinib; gemcitabine; gemifloxacin; gluconic acid; glycolic acid; glycolic acid; glycopeptides such as vancomycin and teicoplanin; griseofulvin; Griseofulvin; guaifenesin; guanethidine; haloperidol; haloprogin; Haloprogin; herbal extract, an alkaloid, a flvanoid, Abafungin; hexylresorcinol; homatropine; homosalate; hydralazine; hydrochlorothiazide; hydrocortisone; hydrocortisone 17-butyrate; hydrocortisone 17-valerate; hydrocortisone 21-acetate; hydromorphone; hydroquinone; hydroquinone monoether; hydroxyzine; hyoscyamine; hypoxanthine; ibuprofen; ichthammol; idarubicin; imatinib; imipramine; imiquimod; indinavir; indomethacin; Iodine; irbesartan; irinotecan; Isavuconazole; Isoconazole; isoetharine; isoproterenol; itraconazole; Itraconazole; kanamycin; ketamine; ketanserin; ketoconazole; ketoprofen; ketotifen; kojic acid; labetalol; lactic acid; lactobionic acid; lactobionic acid; lamivudine; lamotrigine; lansoprazole; lemon myrtle; letrozole; leuprolide; levalbuterol; levofloxacin; lidocaine; lincosamides such as lincomycin and clindamycin; linezolid; lobeline; loperamide; losartan; loxapine; lucensomycin; lysergic diethylamide; macrolides or ketolides such as erythromycin, azithromycin, clarithromycin, and telithromycin; mafenide; malic acid; maltobionic acid; mandelic acid; mandelic acid; maprotiline; mebendazole; mecamylamine; meclizine; meclocycline; memantine; menthol; meperidine; mepivacaine; mercaptopurine; mescaline; metanephrine; metaproterenol; metaraminol; metformin; methadone; methamphetamine; methotrexate; methoxamine; methyl nicotinate; methyl salicylate; methyldopa esters; methyldopamide; methyllactic acid; methylphenidate; metiamide; metolazone; metoprolol; metronidazole; mexiletine; Micafungin; miconazole; Miconazole; midazolam; midodrine; miglustat; minocycline; minoxidil; mirtazapine; mitoxantrone; moexiprilat; molindone; monobenzone; monolactams such as penicillin G, penicillin V, methicillin, oxacillin, cloxacillin, dicloxacillin, nafcillin, ampicillin, amoxicillin, carbenicillin, ticarcillin, meziocillin, piperacillin, azlocillin, temocillin, cepalothin, cephapirin, cephradine, cephaloridine, cefazolin, cefamandole, cefuroxime, cephalexin, cefprozil, cefaclor, loracarbef, cefoxitin, cefmetazole, cefotaxime, ceftizoxime, ceftriaxone, cefoperazone, ceftazidime, cefixime, cefpodoxime, ceftibuten, cefdinir, cefpirome, cefepime, and astreonam; morphine; moxifloxacin; moxonidine; mupirocin; nadolol; naftifine; Naftifine; nalbuphine; nalmefene; naloxone; naproxen; natamycin; Neem Seed Oil; nefazodone; nelfinavir; neomycin; nevirapine; N-guanylhistamine; nicardipine; nicotine; nifedipine; nikkomycins; nimodipine; nisoldipine; nizatidine; norepinephrine; nystatin; nystatin; octopamine; octreotide; octyl methoxycinnamate; octyl salicylate; ofloxacin; olanzapine; Olive leaf extract; olmesartan medoxomil; olopatadine; omeprazole; Omoconazole; ondansetron; Orange oil; oxazolidinones such as linezolid; oxiconazole; Oxiconazole; oxotremorine; oxybenzone; oxybutynin; oxycodone; oxymetazoline; padimate O; palmarosa oil; palonosetron; pantothenic acid; pantoyl lactone; paroxetine; patchouli; pemoline; penciclovir; penicillamine; penicillins; pentazocine; pentobarbital; pentostatin; pentoxifylline; pergolide; perindopril; permethrin; phencyclidine; phenelzine; pheniramine; phenmetrazine; phenobarbital; phenol; phenoxybenzamine; phenpropimorph; phentolamine; phenylephrine; phenylpropanolamine; phenytoin; phosphonomycin; physostigmine; pilocarpine; pimozide; pindolol; pioglitazone; pipamazine; piperonyl butoxide; pirenzepine; Piroctone; piroctone olamine; podofdox; podophyllin; Polygodial; polyhydroxy acids; polymyxin; Posaconazole; pradimicins; pramoxine; pratipexole; prazosin; prednisone; prenalterol; prilocaine; procainamide; procaine; procarbazine; promazine; promethazine; promethazine propionate; propafenone; propoxyphene; propranolol; propylthiouracil; protriptyline; pseudoephedrine; pyrethrin; pyrilamine; pyrimethamine; quetiapine; quinapril; quinethazone; quinidine; quinolones such as nalidixic acid, oxolinic acid, norfloxacin, pefloxacin, enoxacin, ofloxacin, levofloxacin, ciprofloxacin, temafloxacin, lomefloxacin, fleroxacin, grepafloxacin, sparfloxacin, trovafloxacin, clinafloxacin, gatifloxacin, moxifloxacin, sitafloxacin, besifloxacin, besifloxaxin, clintafloxacin, ganefloxacin, gemifloxacin and pazufloxacin; quinupristin; rabeprazole; Ravuconazole; reserpine; resorcinol; retinal; retinoic acid; retinol; retinyl acetate; retinyl palmitate; ribavirin; ribonic acid; ribonolactone; rifampin; rifamycins such as rifampicin (also called rifampin), rifapentine, rifabutin, bezoxazinorifamycin and rifaximin; rifapentine; rifaximin; riluzole; rimantadine; risedronic acid; risperidone; ritodrine; rivasfigmine; rizatriptan; ropinirole; ropivacaine; salicylamide; salicylic acid; salicylic acid; salmeterol; scopolamine; selegiline; Selenium; selenium sulfide; serotonin; Sertaconazole; sertindole; sertraline; sibutramine; sildenafil; silk fibroin; sordarins; sotalol; streptogramins such as quinupristin and daflopristin; streptomycin; strychnine; sulconazole; Sulconazole; sulfabenz; sulfabenzamide; sulfabromomethazine; sulfacetamide; sulfachlorpyridazine; sulfacytine; sulfadiazine; sulfadimethoxine; sulfadoxine; sulfaguanole; sulfalene; sulfamethizole; sulfamethoxazole; sulfanilamide; sulfapyrazine; sulfapyridine; sulfasalazine; sulfasomizole; sulfathiazole; sulfisoxazole; tadalafil; tamsulosin; tartaric acid; tazarotene; Tea tree oil—ISO 4730 (“Oil of Melaleuca, Terpinen-4-ol type”); tegaserol; telithromycin; telmisartan; temozolomide; tenofovir disoproxil; terazosin; terbinafine; Terbinafine; terbutaline; terconazole; Terconazole; terfenadine; tetracaine; tetracycline; tetracyclines such as tetracycline, chlortetracycline, demeclocycline, minocycline, oxytetracycline, methacycline, doxycycline; tetrahydrozoline; theobromine; theophylline; thiabendazole; thioridazine; thiothixene; thymol; tiagabine; timolol; tinidazole; tioconazole; Tioconazole; tirofiban; tizanidine; tobramycin; tocainide; tolazoline; tolbutamide; tolnaftate; Tolnaftate; tolterodine; tramadol; tranylcypromine; trazodone; triamcinolone acetonide; triamcinolone diacetate; triamcinolone hexacetonide; triamterene; triazolam; triclosan; triclosan; Triclosan; triflupromazine; trimethoprim; trimethoprim; trimipramine; tripelennamine; triprolidine; tromethamine; tropic acid; tyramine; undecylenic acid; Undecylenic acid; urea; urocanic acid; ursodiol; vardenafil; venlafaxine; verapamil; vitamin C; vitamin E acetate; voriconazole; Voriconazole; warfarin; xanthine; zafirlukast; zaleplon; zinc pyrithione; Zinc Selenium sulfide; ziprasidone; zolmitriptan; Zolpidem; WS-3; WS-23; menthol; 3-substituted-P-menthanes; N-substituted-P-menthane-3-carboxamides; isopulegol; 3-(1-menthoxy)propane-1,2-diol; 3-(1-menthoxy)-2-methylpropane-1,2-diol; p-menthane-2,3-diol; p-menthane-3,8-diol; 6-isopropyl-9-methyl-1,4-dioxaspiro[4,5]decane-2-methanol; menthyl succinate and its alkaline earth metal salts; trimethylcyclohexanol; N-ethyl-2-isopropyl-5-methylcyclohexanecarboxamide; Japanese mint oil; peppermint oil; menthone; menthone glycerol ketal; menthyl lactate; 3-(1-menthoxy)ethan-I-ol; 3-(I-menthoxy)propan-I-ol; 3-(1-menthoxy)butan-loi; 1-menthylacetic acid N-ethylamide; 1-menthyl-4-hydroxypentanoate; 1-menthyl-3-hydroxybutyrate; N,2,3-trimethyl-2-(-1-methylethyl)-butanamide; n-ethyl-t-2-c-6 nonadienamide; N,N-dimethyl-menthyl succinamide; menthyl pyrrolidone carboxylate; aloe; avocado oil; green tea extract; hops extract; chamomile extract; colloidal oatmeal; calamine; cucumber extract; sodium palmate; sodium palm kernelate; Butyrospermum parkii (i.e., shea butter); menthe piperita (i.e.; peppermint) leaf oil; sericin; pyridoxine (a form of vitamin B6); retinyl palmitate and/or other forms of vitamin A; tocopheryl acetate and/or other forms of vitamin E; lauryl laurate; hyaluronic acid; aloe barbadensis leaf juice powder; Euterpe oleracea (i.e., acai berry) fruit extract; riboflavin (i.e., vitamin B2); thiamin HCl and/or other forms of vitamin BI; ethylenediaminetetraacetic acid (EDTA); citrate; ethylene glycol tetraacetic acid (EGTA); I,2-bis(o-aminophenoxy)ethane-N,N,N′,N′-tetraacetic acid (BAPTA); diethylene triamine pentaacetic acid (DTPA); 2,3-dimercapto-1-propanesulfonic acid (DMPS); dimercaptosuccinic acid (DMSA); α-lipoic acid; salicylaldehyde isonicotinoyl hydrazone (SIH); hexyl thioethylamine hydrochloride (HTA); desferrioxamine; ascorbic acid (vitamin C); cysteine; glutathione; dihydrolipoic acid; 2-mercaptoethane sulfonic acid; 2-mercaptobenzimidazole sulfonic acid; 6-hydroxy-2,5,7,8-tetramethylchroman-2-carboxylic acid; sodium metabisulfite; vitamin E isomers such as α-, β-, γ-, and δ-tocopherols and α-, β-, γ-, and δ-tocotrienols; polyphenols such as 2-tert-butyl-4-methyl phenol, 2-tert-butyl-5-methyl phenol, and 2-tert-butyl-6-methyl phenol; butylated hydroxyanisole (BHA) such as 2-tert-butyl-4-hydroxyanisole and 3-tert-butyl-4-hydroxyanisole; butylhydroxytoluene (BEIT); tert-butylhydroquinone (TBHQ); ascorbyl palmitate; n-propyl gallate; soy extract; soy isoflavones; retinoids such as retinol; kojic acid; kojic dipalmitate; hydroquinone; arbutin; transexamic acid; vitamins such as niacin and vitamin C: azelaic acid; linolenic acid and linoleic acid; placertia; licorice; and extracts such as chamomile and green tea; hydrogen peroxide; zinc peroxide; sodium peroxide; hydroquinone; 4-isopropylcatechol; hydroquinone monobenzyl ether; kojic acid; lactic acid; ascorbyl acid and derivatives such as magnesium ascorbyl phosphate; arbutin; licorice root; dihydroxyacetone (DHA); glyceryl aldehyde; tyrosine and tyrosine derivatives such as malyltyrosine, tyrosine glucosinate, and ethyl tyrosine; phospho-DOPA; indoles and derivatives; glucosamine; N-acetyl glucosamine; glucosamine sulfate; mannosamine; N-acetyl mannosamine; galactosamine; N-acetyl galactosamine; N-acyl amino acid compounds (e.g., N-undecylenoyl-L-phenylalanine); flavonoids such as quercetin, hesperidin, quercitrin, rutin, tangeritin, and epicatechin; CoQIO; vitamin C; hydroxy acids including C2-C30 alpha-hydroxy acids such as glycolic acid, lactic acid, 2-hydroxy butanoic acid, malic acid, citric acid tartaric acid, alpha-hydroxyethanoic acid, hydroxycaprylic acid and the like; beta hydroxy acids including salicylic acid and polyhydroxy acids including gluconolactone (G4); retinoic acid; gamma-linolenic acid; ultraviolet absorber of benzoic acid system such as para-aminobenzoic acid (hereinafter, abbreviated as PABA), PABA monoglycerin ester, N,N-dipropoxy PABA ethyl ester, N,N-diethoxy PABA ethyl ester, N,N-dimethyl PABA ethyl ester, N,N-dimethyl PABA butyl ester, and N,N-dimethyl PABA methyl ester and the like; ultraviolet absorber of anthranilic acid system such as homomenthyl-N-acetyl anthranilate and the like; ultraviolet absorber of salicylic acid system such as amyl salicylate, menthyl salicylate, homomenthyl salicylate, octyl salicylate, phenyl salicylate, benzyl salicylate, p-isopropanol phenyl salicylate and the like; ultraviolet absorber of cinnamic acid system such as octyl cinnamate, ethyl-4-isopropyl cinnamate, methyl-2,5-diisopropyl cinnamate, ethyl-2,4-diisopropyl cinnamate, methyl-2,4-diisopropyl cinnamate, propyl-p-methoxy cinnamate, isopropyl-p-methoxy cinnamate, isoamyl-p-methoxy cinnamate, octyl-p-methoxy cinnamate(2-ethylhexyl-p-methoxy cinnamate), 2-ethoxyethyl-p-methoxy cinnamate, cyclohexyl-p-methoxy cinnamate, ethyl-a-cyano-p-phenyl cinnamate, 2-ethylhexyl-a-cyano-P-phenyl cinnamate, glyceryl-mono-2-ethylhexanoyl-dipara-methoxy-cinnamate, methyl bis(trimethylsiloxane)silylisopentyl trimethoxy cinnamate and the like; 3-(4′-methylbenzylidene)-d, 1-camphor; 3-benzylidene-d,l-camphor; urocanic acid, urocanic acid ethyl ester; 2-phenyl-5-methylbenzoxazole; 2,2′-hydroxy-5-methylphenylbenzotriazole; 2-(2′-hydroxy-5′-t-octylphenyl)benzotriazole; 2-(2′-hydroxy-5′-methylphenylbenzotriazole; dibenzaladine; dianisoylmethane; 4-methoxy-4′-t-butyldibenzoylmethane; 5-(3,3-dimethyl-2-norbornylidene)-3-pentane-2-one; dimorpholinopyridazinone; titanium oxide; particulate titanium oxide; zinc oxide; particulate zinc oxide; ferric oxide; particulate ferric oxide; ceric oxide; inorganic sunscreens such as tianium dioxide and zinc oxide; organic sunscreens such as octyl-methyl cinnamates and derivatives thereof; retinoids; vitamins such as vitamin E, vitamin A, vitamin C (ascorbic acid), vitamin B, and derivatives thereof such as vitamin E acetate, vitamin C palmitate, and the like; antioxidants including alpha hydroxy acid such as glycolic acid, citric acid, lactic acid, malic acid, mandelic acid, ascorbic acid, alpha-hydroxybutyric acid, alpha-hydroxyisobutyric acid, alpha-hydroxyisocaproic acid, atrrolactic acid, alpha-hydroxyisovaleric acid, ethyl pyruvate, galacturonic acid, glucopehtonic acid, glucopheptono-1,4-lactone, gluconic acid, gluconolactone, glucuronic acid, glucurronolactone, glycolic acid, isopropyl pyruvate, methyl pyruvate, mucic acid, pyruvia acid, saccharic acid, saccaric acid 1,4-lactone, tartaric acid, and tartronic acid; beta hydroxy acids such as beta-hydroxybutyric acid, beta-phenyl-lactic acid, beta-phenylpyruvic acid; botanical extracts such as green tea, soy, milk thistle, algae, aloe, angelica, bitter orange, coffee, goldthread, grapefruit, hoellen, honeysuckle, Job's tears, lithospermum, mulberry, peony, puerarua, rice, and safflower; 21-acetoxypregnenolone; alclometasone; algestone; amcinonide; beclomethasone; betamethasone; budesonide; chloroprednisone; clobetasol; clobetansone; clocortolone; cloprednol; corticosterone; cortisone; cortivazol; deflazacort; desonide; desoximetasone; dexamethasone; diflorasone; diflucortolone; difluprednate; enoxolone; fluazacort; flucloronide; flumethasone flunisolide; fluocinolone acetonide; fluocinonide; fluocortin butyl; fluocortolone; fluorometholone; fluperolone acetate; fluprednidene acetate; fluprednisolone; flurandrenolide; fluticasone propionate; formocortal; halcinonide; halobetasol propionate; halometasone; halopredone acetate; hydrocortamate; hydrocortisone; loteprednol etabonate; mazipredone; medrysone; meprednisone; methylprednisolone; mometasone furcate; paramethosone; prednicarbate; prednisolone; prednisolone 25-diethylamino-acetate; prednisolone sodium phosphate; prednisone; prednival; prednylidene; rimexolone; tixocortol; triamcinolone; triamcinolone acetonide; triamcinolone benetonide; triamcinolone hexacetonide; COX inhibitors such as salicylic acid derivatives (e.g., aspirin, sodium salicylate, choline magnesium trisalicylate, salicylate, diflunisal, sulfasalazine and olsalazine); para-aminophenol derivatives such as acetaminophen; indole and indene acetic acids such as indomethacin and sulindac; heteroaryl acetic acids such as tolmetin, dicofenac and ketorolac; arylpropionic acids such as ibuprofen, naproxen, flurbiprofen, ketoprofen, fenoprofen and oxaprozin; anthranilic acids (fenamates) such as mefenamic acid and meloxicam; enolic acids such as the oxicams (piroxicam, meloxicam); alkanones such as nabumetone; diarylsubstituted furanones such as refecoxib; diaryl-substituted pyrazoles such as celecoxib; indole acetic acids such as etodolac; sulfonanilides such as nimesulide; selenium sulfide; sulfur; sulfonated shale oil; salicylic acid; coal tar; povidone-iodine, imidazoles such as ketoconazole, dichlorophenyl imidazolodioxalan, clotrimazole, itraconazoie, miconazole, climbazole, tioconazole, sulconazole, butoconazole, fluconazole, miconazolenitrite; anthralin; piroctone olamine (Octopirox); ciclopirox olamine; anti-psoriasis agents; vitamin A analogs; corticosteroids; and any combinations thereof, including nanoparticulate forms or nanoparticulate compositions comprising any of the aforementioned active agents.

Other definitions for selected terms used herein may be found within the detailed description of the invention and apply throughout. Unless otherwise defined, all other scientific and technical terms used herein have the same meaning as commonly understood to one of ordinary skill in the art to which the invention belongs.

### DETAILED DESCRIPTION

The present inventors have surprisingly found that many of the problems associated with volatility and/or toxicity and/or environmental pollutant potential of halocarbon compounds possessing activity as inhibitors of methanogenesis in ruminants can be ameliorated and/or overcome via stabilization as emulsions or dry powders derived from emulsions comprising said halocarbon compounds. This approach provides the added advantages of addressing the problems associated with dosage variability involved in the use of seaweed as a feed additive, whilst overcoming the problems associated with the environmental impacts of commercial seaweed production and harvesting of seaweed on marine environments.

Methanogens are microorganisms that produce methane as a waste product of their metabolism. They play a crucial role in ruminants' digestive systems. The abomasum, rumen, omasum, and reticulum are the four key components of a ruminant's digestive tract. Food with saliva is first delivered to the rumen, where it is broken down into smaller particles, then to the reticulum, where it is further broken down into smaller particles, and the indigestible particles are sent back to the rumen for rechewing. The rumen is home to the majority of the anaerobic microorganisms that aid in cellulose breakdown. They are responsible for initiating the fermentation process. The animal absorbs fatty acids, vitamins, and nutrient content when it passes partially digested food from the rumen to the omasum, which lowers the pH and thus triggers the release of enzymes to further break down the food, which is then passed to the abomasum, which absorbs the remaining nutrients before excretion. This procedure takes approximately 9-12 hours.

The microbes in ruminant digestive system responsible for methanogenesis include Methanobrevibacter ruminantium, a methanoic, hydrogen utilizing, archaea involved in the creation of methane and Methanosarcina barkeri, a methanoic, hydrogen utilizing, archaea involved in the creation of methane and carbon dioxide.

In these and other species of archaea, methyl-coenzyme M reductase (MCR) catalyses the rate limiting step of methanogenesis (the final step in the biosynthetic methanogenesis process). Higher H₂ emitting bacteria are also correlated with higher methanogenesis. Meanwhile, production of propionate by other bacterial groups consumes ruminal H₂, thus reducing the supply of methanogenesis substrates resulting in lower production of CH₄. Succinate and lactate are also precursors of propionate production. As a result, succinate producing bacteria in the ruminant gastrointestinal biome are correlated with reduced methanogenesis.

Halocarbon compounds including bromoform block the function of corrinoid enzymes and thereby inhibit cobamide-dependent methyl group transfer, required to synthesize methyl-coenzyme M reductase, the key enzyme involved in the final step of methanogenesis, by binding and sequestering the prosthetic group required by MCR to form CH₄. A parallel mechanism of inhibition of methanogenesis by halocarbon compounds has also been suggested in the literature, whereby halogen substituents act as terminal electron acceptors.

Compositions of the present invention may be screened for activity as inhibitors of methanogenesis, utilizing in vitro methods known from the prior art, such as those described herein, or those reported in Ramin, M.; Huhtanen, P. “Development of an in vitro method for determination of methane production kinetics using a fully automated in vitro gas system-A modelling approach.” Anim. FeedSci. Technol. 2012, 174, 190-200; and Chagas, J. C. et al “In Vitro Evaluation of Different Dietary Methane Mitigation Strategies”, Animals 2019, 9, 1120, the contents of which previously published documents are hereby incorporated herein in their entirety.

Within the meaning of the present invention, the anti-methanogenic effect of compositions of the present invention can be measured in the rumen with an artificial rumen system, such as those referenced above, or such as that described herein, or such as that described in T. Hano, J. Gen. Appl. Microbiol., 1993, 39, 35-45, or by in vivo oral administration to ruminants, the contents of which previously published document is hereby incorporated herein in its entirety.

Compositions of the present invention may be evaluated for stability in terms of reduced volatility of the halocarbon compound(s) in the compositions compared to the volatility of the corresponding isolated halocarbon compound(s), as determined by vapour pressure measurements made in accordance with any one of the experimental techniques described in Ambrose, D., “Vapor pressure” Experimental Thermodynamics of Non-reacting Systems, Le Neindre, B.; Vodar, B., Eds. Butterworths: London, 1975; pp 607-656. [ISBN 0408705663]; Carson, A. S., “The measurement of vapor pressure.” Thermochemistry and Its Applications to Chemical and Biochemical Systems, Ribeiro da Silva, M. A. V., Ed., D. Riedel Publishing Company: 1984; pp 127-41. [ISBN 978-9027716989]; Verevkin, S. P., “Phase Changes in Pure Component Systems: Liquids and Gases.” Measurement of the Thermodynamic Properties of Multiple Phases, Weir, R. D.; de Loos, T. W., Eds. Elsevier: Amsterdam, 2005; pp 5-30. [ISBN 0444519777]; or Delle Site, A., “The vapor pressure of environmentally significant organic chemicals: a review of methods and data at ambient temperature.” Journal of Physical and Chemical Reference Data 1997, 26, (1), 157-193, the contents of which previously published documents are hereby incorporated herein in their entirety.

The invention will now be described in greater detail, by way of example, with reference to the following examples in which a range of approaches have been developed for the stabilisation of halocarbon compounds with antimethanogenic activity, utilising formulation ingredients that are food, or food additives, or food derived, or Generally Recognized As Safe (GRAS) in accordance with the relevant legislation and regulations applied by the USFDA.

In some of these approaches, milk proteins derived from whole milk or milk solids are utilised. Without wishing to be bound by theory, the present inventors believe that globular proteins such as those found in milk act as emulsifiers and contribute to the stability of the formulations of some embodiments of the invention by binding to the antimethanogenic halocarbon compounds in these formulations.

### Examples

### Materials

Fresh pasteurized milk (PURA™), whole milk powder (Devondale™), infant formula (Aptamil Gold+™), gelatin, xanthan/xanthum gum (Macro™), and soy lecithin (Macro™) were purchased from a local supermarket; bromoform (CHBr₃), Tween and nitric acid (HNO₃) (analytical grade) were purchased from Sigma Aldrich; EtOH (95%) was purchased from CSR; MCT (caprylic/capric triglyceride) was purchased from Australian Wholesale Oils; high purity water was used.

### Preparation of Emulsions

Emulsion 1: Bromoform (195 mg), EtOH (75 ml), and lecithin (1500 mg) were combined with whole fresh milk to a total volume of 1000 ml with mechanical stirring at room temperature.
Emulsion 2: Bromoform (195 mg), EtOH (75 ml), and whole milk powder (200 g) were combined with high purity water to a total volume of 1000 ml with mechanical stirring at room temperature.

A notable reduction of bromoform as detected by smell was observed upon formation of both emulsions. The emulsions were stored at 2-5° C. for 7 days and an aliquot of each solution was taken for assay on each day.

### Preparation of Dry Powders

50 ml of each of Emulsions 1 and 2 were subjected to spray drying to form dry powder samples for analysis.

### Characterisation

Aliquots of the emulsions were diluted 5× (2 parts emulsion with 8 parts diluent) with a 1:1 solution of 3% nitric acid (in high purity water) and ethanol (thus the concentration of nitric acid in the 1:1 solution was 1.5%) as diluent, and analysed by Inductively Coupled Plasma-Atomic Emission Spectroscopy (ICP-AES) to determine the bromine content of the emulsions on each day. Concentration of bromine was determined against high purity calibration standards. Ethanol (20% in high purity water) was spiked into calibration standards to ensure external calibration was comparable. Control samples containing all components corresponding to each of emulsions 1 and 2 but without any bromoform added were also analyzed, and these control samples returned results below the limit of detection of the ICP-AES apparatus.

Samples of the dry powder (200 mg) were taken each day and re-emulsified in 10 ml of 20% ethanol in high purity water before being subjected to the same ICP-AES analysis used to analyse the emulsions.

### Results

TABLE 1
Bromoform Stability in Emulsion Compositions

| | T = Days | | | | | | |
|---|---|---|---|---|---|---|---|
| | Calculated CHBr₃ | T = 0 | T = 1 | T = 2 | T = 5 | T = 7 | T = 14 |
| | Conc. | CHBr₃ | CHBr₃ | CHBr₃ | CHBr₃ | CHBr₃ | CHBr₃ |
| | (mg/kg) | (mg/kg) | (mg/kg) | (mg/kg) | (mg/kg) | (mg/kg) | (mg/kg) |
| Emulsion 1 | 195 | 180 ± 14 | 161 ± 17 | 158 ± 13 | 150 ± 19 | 165 ± 9.1 | 163 ± 14 |
| Emulsion 2 | 195 | 185 ± 18 | 179 ± 14 | 189 ± 16 | 184 ± 8 | 190 ± 15 | 185 ± 16 |

The results in Table 1 demonstrate that both emulsions have maintained stable levels of bromoform under the conditions tested.

TABLE 2
Bromoform Stability in Dry Powder Compositions

| | T = Days | | | | | |
|---|---|---|---|---|---|---|
| | Mass of | | Calculated CHBr₃ | | | |
| | Powder | Recovery | Conc. Solid (mg/kg) | T = 0 | T = 2 | T = 7 |
| | Recovered | of Powder | based on T = 7 | CHBr₃ | CHBr₃ | CHBr₃ |
| | (g) | (%) | emulsion data | (mg/kg) | (mg/kg) | (mg/kg) |
| Emulsion 1 | 4.43 g | 68.15 | 1270 | 785 ± 80 | 792 ± 80 | 769 ± 75 |
| Emulsion 2 | 8.25 g | 82.5 | 950 | 849 ± 85 | 860 ± 85 | 845 ± 80 |

| | T = Days | | | | | |
|---|---|---|---|---|---|---|
| | T = 30 | T = 60 | T = 90 | T = 180 | T = 240 | T = 360 |
| | CHBr₃ | CHBr₃ | CHBr₃ | CHBr₃ | CHBr₃ | CHBr₃ |
| | (mg/kg) | (mg/kg) | (mg/kg) | (mg/kg) | (mg/kg) | (mg/kg) |
| Emulsion 1 | 765 ± 60 | 770 ± 65 | 750 ± 69 | 745 ± 82 | 739 ± 43 | 725 ± 48 |
| Emulsion 2 | 882 ± 91 | 880 ± 75 | 840 ± 71 | 842 ± 79 | 835 ± 62 | 826 ± 65 |

The results in Table 2 demonstrate that although some bromoform was lost during spray drying (calculated bromoform concentration based on T=7 emulsion data vs T=0 data for the dry powders), dry powders derived from both emulsions have maintained stable levels of bromoform for up to 360 days under the conditions tested. Stability of samples of dry powders prepared from emulsion 1 and emulsion 2 were also validated at T=360 days via GCMS.

### Preparation of Further Compositions

A series of further embodiments of compositions of the invention were prepared as provided in Table 3.

TABLE 3
Stabilised Halocarbon Composition Formulations

| Formulation | | |
|---|---|---|
| ID | Type | Preparation |
| R8-1 | Full cream milk | Stock (142 mg/mL Bromoform) Prepared: 38 mL MCT, 0.25- |
| | carrier | 1 mL of Ethanol, 2 mL of Bromoform. Diluted with MCT to target |
| | (including | concentration for trial. Various concentrations prepared-milk |
| | emulsifiers) | only, 1.5 mg/mL bromoform, 0.75 mg/ml and 0.15 mg/ml |
| R8-2 | Milk powder | 29 g milk powder/200ml water solution prepared. Stock |
| | re-emulsified | (142 mg/mL Bromoform) Prepared: 38 mL MCT, 0.25-1 mL of |
| | | Ethanol, 2 mL of Bromoform. Small volume of spike added to |
| | | the milk powder solution to target concentration for trial. |
| | | Various concentrations prepared-milk powder solution only, |
| | | 1.5 mg/mL bromoform, 0.75 mg/mL and 0.15 mg/mL |
| R8-3 | Double | 58 g milk powder/200 ml water solution prepared. Stock |
| | solid:milk ratio | (142 mg/mL Bromoform) Prepared: 38 mL MCT, 0.25-1 mL of |
| | | Ethanol, 2 mL of Bromoform. Small volume of spike added to |
| | | the milk powder solution to target concentration for trial. |
| | | Various concentrations prepared-milk powder solution only, |
| | | 1.5 mg/mL bromoform, 0.75 mg/ml and 0.15 mg/ml |
| R8-4 | Infant formula | 4 scoops of infant formula powder into 200 mL hot water |
| | | prepared. Stock (142 mg/mL Bromoform) Prepared: 38 mL |
| | | MCT, 0.25-1 mL of Ethanol, 2 mL of Bromoform. Small volume |
| | | of spike added to the infant formula powder solution to target |
| | | concentration for trial. Various concentrations prepared-infant |
| | | formula powder solution only, 1.5 mg/mL bromoform, |
| | | 0.75 mg/ml and 0.15 mg/mL |
| R8-5 | MCT | Stock (144.5 mg/mL Bromoform) Prepared: 25 mL MCT, 0.25- |
| | | 0.75 mL of Ethanol, 2 mL of Bromoform. Diluted with MCT to |
| | | target concentration for trial |
| R8-7 | gelatin solid- | 25 g Milk Powder, 20 g of Gelatin Powder, 175 ml Water, |
| | clear | Bromoform to Dose. Prep Strategy Milk Powder and Water |
| | | first. Then Bromoform. Then Gelatin. Mix and pour into moulds |
| R8-9 | liquid double | Stock (142 mg/mL Bromoform) Prepared: 38 mL MCT, 0.25- |
| | milk ratio | 1 mL of Ethanol, 2 mL of Bromoform. 58g milk powder/200 mL |
| | | water milk powder solution prepared. Milk powder solution |
| | | spiked with stock solution to dose. Various concentrations |
| | | prepared-0 to 3 mg/ml range |
| R8-10 | solid double | Stock (142 mg/mL Bromoform) Prepared: 38 mL MCT, 0.25- |
| | milk ratio | 1 mL of Ethanol, 2 mL of Bromoform. 58g milk powder/200 mL |
| | | water, 20g gelatin milk powder gelatin solution prepared. Milk |
| | | powder gelatin solution spiked with stock solution to dose. |
| | | Various concentrations prepared-0 to 3 mg/mL range |
| R8-11 | MCT oil | Stock (142 mg/mL Bromoform) Prepared: 38 mL MCT, 0.25- |
| | | 1 mL of Ethanol, 2 mL of Bromoform. |
| R8-12 | water-soluble | Water (350 mL) and Lecithin (3.5 mL) prepared. Bromoform |
| | | (0.5 mL) added to prepare a stock of this solution. This was |
| | | then diluted using a matched lecithin/water solution to hit dose. |
| R8-13 | water-soluble | Water (350 mL) and Lecithin (3.5 mL) and Xanthum Gum (1.5 g) |
| | | prepared. Bromoform (0.5 mL) added to prepare a stock of this |
| | | solution. This was then diluted using a matched lecithin/water |
| | | solution to hit dose. |
| R8-14 | water-soluble | Water (350 mL) and Lecithin (3.5 mL) prepared. Bromoform |
| | | (0.2 mL) was added to MCT (30 mL). This MCT/Bromoform |
| | | Solution was then spiked to hit dose in water/lecitihin solution. |
| R8-15 | water-soluble | Water (330 ml) and Lecithin (5 g) and Xanthum Gum (1 g) |
| | | prepared. Bromoform (2 mL) was added to MCT (38 mL). This |
| | | MCT/Bromoform Solution was then spiked to hit dose in |
| | | water/lecitihin/gum solution. |
| R8-17 | water-soluble | Water (350 mL) and Lecithin (3.5 mL) and Xanthum Gum (1.5 g) |
| | | prepared. Bromoform (2 mL) was added to MCT (38 mL). This |
| | | MCT/Bromoform Solution was then spiked to hit dose in |
| | | water/lecitihin/gum solution. |
| R8-18 | water-soluble | Water (350 mL) and Tween (0.5%/v) prepared. Bromoform was |
| | Tween | added to achieve target concentration. |
| R8-19 | water-soluble | Water (350 mL) and Tween (0.01%) and Xanthum Gum (1-5%) |
| | Tween | prepared. Bromoform (2 mL) was added to MCT (30 mL). This |
| | | MCT/Bromoform Solution was then spiked to hit dose in |
| | | water/lecithin/gum solution. |
| R8-9i | liquid double | 53 g Milk Powder, Made up to 185 g with hot water = Milk |
| | milk ratio | Solution Stock of bromoform made with this milk solution via |
| | | the addition of bromoform and then diluted with milk solution to |
| | | dose |
| R8-10i | solid double | 50 g Milk Powder, 20 g Gelatin Powder made up to 175 g of |
| | milk ratio | water. Bromoform added to spike concentration. |
| R8-17i | water-soluble | Water (350 mL) and Lecithin (3.5 mL) and Xanthum Gum (1.5 g) |
| | | prepared. Bromoform (0.2 mL) was added to MCT (30 mL). This |
| | | MCT/Bromoform Solution was then spiked to hit does in |
| | | water/lecitihin/gum solution. |

All formulations were prepared via addition of the ingredients in the order indicated in Table 3, with mechanical stirring at room temperature (unless otherwise indicated).

### Slow-Release (Solid) Formulation

A general procedure for the preparation of a slow-release solid formulation which is suitable for incorporation into a bolus was developed, involving the following preparation steps. Examples of these formulations are R8-10 and R8-10i.

A stock MCT/Bromoform solution is prepared (144.5 mg/mL Bromoform): 25 mL MCT, 0.25-0.75 mL of Ethanol, 2 mL of Bromoform. A paste of milk powder (100 g) is prepared via careful addition of water with stirring. A measured aliquot of stock MCT/Bromoform solution is added to the paste with stirring. Additional water and then gelatin (100 g) are added to the mixture with stirring to achieve a homogeneous mixture. The mixture is then poured into moulds and allowed to set. The dosage of Bromoform in each slow-release solid pellet, pill or tablet is determined by dividing the total amount of Bromoform in the aliquot of stock solution used in their preparation by the number of solid pellets, pills or tablets produced.

When placed in water at room temperature, the slow-release solid formulations prepared in accordance with this procedure do not completely dissolve after 21 days.

### Antimethanogenic Activity

To investigate the antimethanogenic activity of the stabilised halocarbon composition formulations of the invention, in vitro batch screening of the formulations was conducted in accordance with the procedures described in Durmic et al (Durmic, Z, Hutton, P, Revell, D K, Emms, J, Hughes, S, Vercoe, P E (2010) “In vitro fermentative traits of Australian woody perennial plant species that may be considered as potential sources of feed for grazing ruminants.” Animal Feed Science and Technology 160, 98-109, & Durmic, Z, Moate, PJ, Eckard, R, Revell, DK, Williams, R, Vercoe, PE (2014) “In vitro screening of selected feed additives, plant essential oils and plant extracts for rumen methane mitigation.” Journal of the Science of Food and Agriculture 94, 1191-1196. 10.1002/jsfa.6396), the contents of which documents are hereby incorporated in their entirety.

The methodology mimics processes in the rumen. Ruminal fluid and digesta are collected from fistulated donor sheep and mixed with the substrate containing treatment in a fermentation vessel. The substrate is then fermented by rumen microbes over a period of time (as it would be in the animal), and at the end of incubation, microbial activity is assessed by measuring parameters (i.e. pH) and end-products such microbial gas, CH4, volatile fatty acids (VFA) and NH₃. The IVFT is a short-term (i.e. 24 h), enclosed incubation in a test tube or vial, that is suitable for screening antimethanogenic activity.

A series of in vitro batch fermentation assays (IVFT) were performed on samples of substrate in the absence of halocarbon (formulation carriers only) and in the presence of a range of different formulations having a range of halocarbon dosage.

The experiments were conducted using rumen fluid from standardized, fistulated sheep donors. Animals and their use were approved under UWA AEC protocol RA/3/100/1622. Sheep were chosen in preference to cattle because they are less expensive, and at this stage of evaluation, the differences in rumen fluid and activity of microbes between the two ruminant species are negligible.

A high fibre feed oaten chaff was used as a fermentation substrate and as Control throughout experiments. Oaten chaff was selected as a common supplemental feed in sheep, but also as a high-fibre, and therefore highly methanogenic substrate, and because it does not contain any plant secondary compounds that could interfere with the assay.

The IVFT experiments were setup and run as described earlier—as per Durmic et al. Fermentation substrate was added to fermentation vials one day prior the experiment and left in the anaerobic chamber to get rid of the oxygen. On the experimental day, rumen contents were collected and strained, mixed with buffer and used to inoculate vials. The treatments were compared directly to their respective batch Controls (feed substrate only) to understand the effect of total product (carrier+bioactive), but effect of carrier itself was also considered and to take into account any influence of carrier on parameters measured, and the effect was also compared to the effect of formulation without bromoform (carrier only).

Formulations were examined, in a first series of experiments at three different doses—i.e. carrier control—no bromoform (A), low dose (D), medium dose (C) and high dose (B) of bromoform (Table 4). Bromoform doses were based on available literature data using A. taxiformis to reduce ruminant methane emissions.

The treatments were included in the fermentation after the substrate (oaten chaff, ground to 1 mm and 0.5 g per vial) and fermentation fluid (50 mL per vial) were added to a 120 mL serum vial. The treatments (100 μL for liquids and 100 mg for gel) were pipetted directly into the vial, before it was sealed and crimped. Vials were incubated in a shaking incubator, in a vertical position, with shaking at 50 rpm, at 39° C. for 24 h. All the measurements and statistical analyses were conducted as outlined in Durmic et al.

The addition of carriers (control treatments ‘A’) tended to promote gas production (Table 4). The majority of the control treatments had no to little impact on total gas production when compared to substrate only. Only four control treatments significantly (P<0.05) reduced gas, particularly at the higher doses, with up to 10% reduction recorded with R8-5-B.

The addition of carriers alone (control treatments A) did not reduce CH₄, but there were many bromoform treatments that significantly reduced CH₄ concentrations and production when compared to control treatments. The most potent effects were seen with medium and high doses (C and D), causing up to 98% reduction. In some, these strong effects on CH₄ were also accompanied with some small, but significant reductions in overall gas production.

TABLE 4
IVFT Results

| | | Bromoform | Final | | |
|---|---|---|---|---|---|
| | | dose | conc | % | |
| Formulation/ | | (mg) | (g/kg | Bioactive/ | % CH4 |
| Treatment | Dose | per vial | DMI) | DMI | reduction |
| R8-2-A | None | 0 | | | |
| R8-2-D | Low | 0.015 | 0.03 | 0.003 | 4 |
| R8-2-C | Med | 0.075 | 0.15 | 0.015 | 97 |
| R8-2-B | High | 0.15 | 0.3 | 0.03 | 98 |
| R8-3-A | None | 0 | | | |
| R8-3-D | Low | 0.015 | 0.03 | 0.003 | 3 |
| R8-3-C | Med | 0.075 | 0.15 | 0.015 | 95 |
| R8-3-B | High | 0.15 | 0.3 | 0.03 | 97 |
| R8-4-A | None | 0 | | | |
| R8-4-D | Low | 0.015 | 0.03 | 0.003 | 7 |
| R8-4-C | Med | 0.075 | 0.15 | 0.015 | 11 |
| R8-4-B | High | 0.15 | 0.3 | 0.03 | 14 |
| R8-5-A | None | 0 | | | |
| R8-5-D | Low | 0.015 | 0.03 | 0.003 | 9 |
| R8-5-C | Med | 0.075 | 0.15 | 0.015 | 95 |
| R8-5-B | High | 0.15 | 0.3 | 0.03 | 93 |
| R8-7-A | None | 0 | | | |
| R8-7-D | Low | 0.015 | 0.03 | 0.003 | 8 |
| R8-7-C | Med | 0.075 | 0.15 | 0.015 | 19 |
| R8-7-B | High | 0.15 | 0.3 | 0.03 | 97 |

No bromoform (A), low dose (D), medium dose (C) and high dose (B) of bromoform;
DMI = Dry Matter Incubated.
Table 4 formulations were tested 5 days after preparation.

The formulations R8-3, R8-7, R8-2, and R8-5, at medium to high doses are capable of significantly reducing CH₄ (by up to 98%), with only small to moderate reduction in gas when compared to control (up to 10%). Increasing the dose after ‘medium’ did not appear to provide any further significant improvement in effect on reducing CH₄, except in R8-7 where the high dose was nearly five times more potent than the medium dose. The top three formulations included an oil (R8-5), a solid (R8-7), and a liquid (R8-3).

A further series of IVFT experiments was performed, across a broader range of dosages ranging from no bromoform (A), to very low bromoform at 0.02 mg (B), and across a spectrum; 0.04 mg (C), 0.06 mg (D), 0.08 mg (E), 0.1 mg (F), 0.12 mg (G), 0.14 mg (H), 0.2 mg (I), through to very high bromoform at 0.3 mg (J), (Table 5).

TABLE 5
IVFT Results

| | | Bromoform | Final | | |
|---|---|---|---|---|---|
| | | dose | conc | % | |
| Formulation/ | | (mg) | (g/kg | Bioactive/ | % CH4 |
| Treatment | Dose | per vial | DMI) | DMI | reduction |
| R8-9-A | None | 0 | 0.00 | 0 | |
| R8-9-B | very low | 0.02 | 0.04 | 0.004 | 26 |
| R8-9-C | | 0.04 | 0.08 | 0.008 | 23 |
| R8-9-D | | 0.06 | 0.12 | 0.012 | 98 |
| R8-9-E | | 0.08 | 0.16 | 0.016 | 98 |
| R8-9-F | | 0.1 | 0.20 | 0.02 | 98 |
| R8-9-G | | 0.12 | 0.24 | 0.024 | 97 |
| R8-9-H | | 0.14 | 0.28 | 0.028 | 98 |
| R8-9-1 | | 0.2 | 0.40 | 0.04 | 98 |
| R8-9-J | very high | 0.3 | 0.60 | 0.06 | 98 |
| R8-10-A | 0 | 0 | 0.00 | 0 | |
| R8-10-B | very low | 0.02 | 0.04 | 0.004 | 11 |
| R8-10-C | | 0.04 | 0.08 | 0.008 | 76 |
| R8-10-D | | 0.06 | 0.12 | 0.012 | 95 |
| R8-10-E | | 0.08 | 0.16 | 0.016 | 94 |
| R8-10-F | | 0.1 | 0.20 | 0.02 | 98 |
| R8-10-G | | 0.12 | 0.24 | 0.024 | 99 |
| R8-10-H | | 0.14 | 0.28 | 0.028 | 98 |
| R8-10-1 | | 0.2 | 0.40 | 0.04 | 98 |
| R8-10-J | very high | 0.3 | 0.60 | 0.06 | 98 |
| R8-11-A | 0 | 0 | 0.00 | 0 | |
| R8-11-B | very low | 0.02 | 0.04 | 0.004 | 14 |
| R8-11-C | | 0.04 | 0.08 | 0.008 | 95 |
| R8-11-D | | 0.06 | 0.12 | 0.012 | 96 |
| R8-11-E | | 0.08 | 0.16 | 0.016 | 97 |
| R8-11-F | | 0.1 | 0.20 | 0.02 | 96 |
| R8-11-G | | 0.12 | 0.24 | 0.024 | 96 |
| R8-11-H | | 0.14 | 0.28 | 0.028 | 96 |
| R8-11-I | | 0.2 | 0.40 | 0.04 | 97 |
| R8-11-J | very high | 0.3 | 0.60 | 0.06 | 97 |

DMI = Dry Matter Incubated.
Table 5 formulations were tested 14 days after preparation.

Formulations R8-9, R8-10 and R8-11 showed impressive anti methanogenic activity, with 95-99% reductions in methane production even at relatively low doses.

A further series of IVFT experiments was performed, to test the efficacy of four different water-soluble bromoform formulations, R8-12, R8-13, R8-14, and R8-15, with no bromoform (A), low dose bromoform (D), medium dose bromoform (C), high dose bromoform (B), and the effects of high dose bromoform on pelleted substrate (B*), (Table 6).

Traditionally, and in the foregoing experiments, oaten chaff is used as the substrate in the in IVFT assays, however one of the aims of the experiment in Table 6 was to assess the effect of the formulations of the invention on a feed pellet commonly used commercially and in live animal trials as the substrate. The high dose (B) of the four water-soluble formulations were tested in the 24-hour batch culture with a pellet substrate (101 pellet, Macco Feeds, Williams WA). The formulations were added to the vial as described above in accordance with the preceeding IVFT experiments, with 0.5 g ground pellets used in place of oaten chaff.

TABLE 6
IVFT Results

| | | Bromoform | Final | | |
|---|---|---|---|---|---|
| | | dose | conc | % | |
| Formulation/ | | (mg) | (g/kg | Bioactive/ | % CH4 |
| Treatment | Dose | per vial | DMI) | DMI | reduction |
| R8-12-A | none | 0 | 0 | 0 | 0 |
| R8-12-B | high | 0.15 | 0.3 | 0.03 | 98 |
| R8-12-C | med | 0.075 | 0.15 | 0.015 | 98 |
| R8-12-D | low | 0.015 | 0.03 | 0.003 | 6 |
| R8-12-B* | high | 0.15 | 0.3 | 0.03 | 98 |
| R8-13-A | none | 0 | 0 | 0 | 0 |
| R8-13-B | high | 0.15 | 0.3 | 0.03 | 98 |
| R8-13-C | med | 0.075 | 0.15 | 0.015 | 15 |
| R8-13-D | low | 0.015 | 0.03 | 0.003 | 0 |
| R8-13-B* | high | 0.15 | 0.3 | 0.03 | 98 |
| R8-14-A | none | 0 | 0 | 0 | 0 |
| R8-14-B | high | 0.15 | 0.3 | 0.03 | 97 |
| R8-14-C | med | 0.075 | 0.15 | 0.015 | 97 |
| R8-14-D | low | 0.015 | 0.03 | 0.003 | 5 |
| R8-14-B* | high | 0.15 | 0.3 | 0.03 | 98 |
| R8-15-A | none | 0 | 0 | 0 | 0 |
| R8-15-B | high | 0.15 | 0.3 | 0.03 | 98 |
| R8-15-C | med | 0.075 | 0.15 | 0.015 | 98 |
| R8-15-D | low | 0.015 | 0.03 | 0.003 | 1 |
| R8-15-B* | high | 0.15 | 0.3 | 0.03 | 98 |

*Feed pellet substrate.
DMI = Dry Matter Incubated.
Table 6 formulations were tested 5 days after preparation.

The high (B) and medium (C) doses of all formulations significantly decreased both CH₄ concentration and production by 97-98% compared to the oaten chaff control (P<0.05). The low dose of formulation R8-12 (R8-12-D), reduced methane by a small but significant amount compared to the oaten chaff control.

None of the formulations tested had an impact on total gas production when compared to pellets only. The high doses of formulations R8-12-B*, R8-13-B*, R8-14-B* and R8-15-B* significantly reduced CH₄ concentration and production by 98% compared to pellets only (P<0.05), a similar reduction to when these formulations were tested with an oaten chaff substrate.

A further series of IVFT experiments was performed, across a broader range of dosages ranging from no bromoform control (A), to very low bromoform at 0.02 mg (B), and across a spectrum; 0.04 mg (C), 0.06 mg (0), 0.08 mg (E), 0.1 mg (F), 0.12 mg (G), 0.14 mg (H), 0.2 mg (1), through to very high bromoform at 0.3 mg (J). The tests were performed on Formulation R8-17, a water-soluble formulation, Formulation R8-18, a surfactant-based formulation without an oil carrier, and Formulation R8-19, a formulation including both a surfactant and an oil carrier (Table 7).

The assays were 24-hour batch cultures run and analysed as detailed in Durmic et al. (2014). The treatments (100 μL) were included in the fermentation after the substrate (0.5 g oaten chaff per vial) and fermentation fluid (50 mL per vial) were added. Vials were sealed and incubated in a shaking incubator, in a vertical position, with shaking at 50 rpm, at 39° C. for 24 h. After incubation, total gas pressure and methane concentration in the headspace gas was measured, and samples of the fermentation fluid were analysed for VFAs and NH₃.

TABLE 7
IVFT Results

| | | Bromoform | Final | | |
|---|---|---|---|---|---|
| | | dose | conc | % | |
| Formulation/ | | (mg) | (g/kg | Bioactive/ | % CH4 |
| Treatment | Dose | per vial | DMI) | DMI | reduction |
| R-17-A | none | 0 | 0.00 | 0 | |
| R-17-B | very low | 0.02 | 0.04 | 0.004 | 97 |
| R-17-C | | 0.04 | 0.08 | 0.008 | 98 |
| R-17-D | | 0.06 | 0.12 | 0.012 | 98 |
| R-17-E | | 0.08 | 0.16 | 0.016 | 98 |
| R-17-F | | 0.1 | 0.20 | 0.02 | 98 |
| R-17-G | | 0.12 | 0.24 | 0.024 | 98 |
| R-17-H | | 0.14 | 0.28 | 0.028 | 98 |
| R-17-1 | | 0.2 | 0.40 | 0.04 | 98 |
| R-17-J | very high | 0.3 | 0.60 | 0.06 | 98 |
| R-18-A | 0 | 0 | 0.00 | 0 | |
| R-18-B | very low | 0.02 | 0.04 | 0.004 | 4 |
| R-18-C | | 0.04 | 0.08 | 0.008 | −9 |
| R-18-D | | 0.06 | 0.12 | 0.012 | 8 |
| R-18-E | | 0.08 | 0.16 | 0.016 | 2 |
| R-18-F | | 0.1 | 0.20 | 0.02 | −3 |
| R-18-G | | 0.12 | 0.24 | 0.024 | 0 |
| R-18-H | | 0.14 | 0.28 | 0.028 | −3 |
| R-18-1 | | 0.2 | 0.40 | 0.04 | 6 |
| R-18-J | very high | 0.3 | 0.60 | 0.06 | 20 |
| R-19-A | 0 | 0 | 0.00 | 0 | |
| R-19-B | very low | 0.02 | 0.04 | 0.004 | 19 |
| R-19-C | | 0.04 | 0.08 | 0.008 | 13 |
| R-19-D | | 0.06 | 0.12 | 0.012 | 97 |
| R-19-E | | 0.08 | 0.16 | 0.016 | 99 |
| R-19-F | | 0.1 | 0.20 | 0.02 | 97 |
| R-19-G | | 0.12 | 0.24 | 0.024 | 98 |
| R-19-H | | 0.14 | 0.28 | 0.028 | 98 |
| R-19-1 | | 0.2 | 0.40 | 0.04 | 98 |
| R-19-J | very high | 0.3 | 0.60 | 0.06 | 98 |

DMI = Dry Matter Incubated.
Table 7 formulations were tested 5 days after preparation.

The addition of carriers R8-17-A and R8-19-A had no effect on total gas production compared to the oaten chaff controls (p<0.05). Additionally, at all doses tested, the R8-17 and R8-19 formulations did not alter total gas production compared to their respective oaten chaff controls or carrier only treatments (p<0.05). The carrier only treatment R8-18-A had no significant effect on total gas production compared to the oaten chaff control, and the R8-18 treatments R8-18-F, -G, -H, -I and -J did not significantly alter total gas production compared to the oaten chaff control or the carrier only.

Compared to the oaten chaff control, the carrier only treatment R8-17-A increased methane concentration by 7% and methane production by 9% (p<0.05). The carrier only treatment R8-18-A increased (p<0.05) methane production by 8%.

All nine doses of the R8-17 formulation (B to J) reduced (p<0.05) methane production and methane concentration by 97-98% compared to the oaten chaff control, with no significant differences in reduction between the nine treatments.

All nine doses of the R8-19 formulations (B to J) reduced methane production and methane concentration compared to the oaten chaff control (p<0.05). The two lowest doses, B and C, resulted in 12-19% reductions in methane concentration and production. The further 7 doses (D to J) significantly reduced both methane concentration and production by at least 97% compared to the oaten chaff control.

Formulation R8-18 demonstrated less antimethanogenic activity compared to the other formulations, with treatment R8-18-D causing a small but significant reduction in methane concentration and production of 8%. The highest dose R8-18-J caused an 18% and 20% reduction in methane concentration and production respectively when compared to the oaten chaff control.

There were no significant changes in total VFA concentrations with any treatments (p<0.05). The NH₃ concentration was reduced by the six low to medium doses of formulations R8-17 (p<0.05), dropping to as low as 26 mg/mL compared to 57 mg/mL for the oaten chaff control, a 54% reduction. Formulation R8-19 also significantly reduced NH₃, with treatment R8-19-E reducing NH₃ by 70% compared to the oaten chaff control. None of the R8-18 formulations significantly affected NH₃ concentrations. These results indicate that the formulations R8-17 and R8-19 are capable of inhibiting methane production without adversely affecting overall fermentation.

A particularly surprising result is the significant decrease in NH₃ concentration seen with formulations R8-17 and R8-19. Reduced ruminal NH₃ improves nitrogen utilisation, potentially by the inhibition of hyperammonia-producing bacteria (HAP) that deaminate amino acids to ammonia that is then subsequently lost across the rumen wall.

A further series of IVFT experiments was performed, across a broader range of dosages ranging from no bromoform control (A), to very low bromoform at 0.02 mg (B), and across a spectrum; 0.04 mg (C), 0.06 mg (D), 0.08 mg (E), 0.1 mg (F), 0.12 mg (G), 0.14 mg (H), 0.2 mg (I), through to very high bromoform at 0.3 mg (J).

The tests were performed on Formulations R8-11, R8-9i, R8-10i, and R8-17i.

The same procedure was followed as for the preceding IVFT experiments, using oaten chaff as substrate, but substituting cow rumen fluid for sheep rumen fluid in order to assess the performance of the formulations in the presence of a different microbial cohort (Table 8).

TABLE 8
IVFT Results (Cow Rumen Fluid)

| | | Bromoform | Final | | |
|---|---|---|---|---|---|
| | | dose | conc | % | |
| Formulation/ | | (mg) | (g/kg | Bioactive/ | % CH4 |
| Treatment | Dose | per vial | DMI) | DMI | reduction |
| R-11-A | none | 0 | 0.00 | 0 | |
| R-11-B | very low | 0.02 | 0.04 | 0.004 | 98 |
| R-11-C | | 0.04 | 0.08 | 0.008 | 99 |
| R-11-D | | 0.06 | 0.12 | 0.012 | 99 |
| R-11-E | | 0.08 | 0.16 | 0.016 | 99 |
| R-11-F | | 0.1 | 0.20 | 0.02 | 99 |
| R-11-G | | 0.12 | 0.24 | 0.024 | 99 |
| R-11-H | | 0.14 | 0.28 | 0.028 | 99 |
| R-11-1 | | 0.2 | 0.40 | 0.04 | 99 |
| R-11-J | very high | 0.3 | 0.60 | 0.06 | 99 |
| R-9i-A | 0 | 0 | 0.00 | 0 | |
| R-9i-B | very low | 0.02 | 0.04 | 0.004 | 98 |
| R-9i-C | | 0.04 | 0.08 | 0.008 | 99 |
| R-9i-D | | 0.06 | 0.12 | 0.012 | 99 |
| R-9i-E | | 0.08 | 0.16 | 0.016 | 99 |
| R-9i-F | | 0.1 | 0.20 | 0.02 | 99 |
| R-9i-G | | 0.12 | 0.24 | 0.024 | 99 |
| R-9i-H | | 0.14 | 0.28 | 0.028 | 99 |
| R-9i-I | | 0.2 | 0.40 | 0.04 | 92 |
| R-9i-J | very high | 0.3 | 0.60 | 0.06 | 99 |
| R-10i-A | 0 | 0 | 0.00 | 0 | |
| R-10i-B | very low | 0.02 | 0.04 | 0.004 | 99 |
| R-10i-C | | 0.04 | 0.08 | 0.008 | 94 |
| R-10i-D | | 0.06 | 0.12 | 0.012 | 99 |
| R-10i-E | | 0.08 | 0.16 | 0.016 | 99 |
| R-10i-F | | 0.1 | 0.20 | 0.02 | 99 |
| R-10i-G | | 0.12 | 0.24 | 0.024 | 99 |
| R-10i-H | | 0.14 | 0.28 | 0.028 | 99 |
| R-10i-I | | 0.2 | 0.40 | 0.04 | 99 |
| R-10i-J | very high | 0.3 | 0.60 | 0.06 | 99 |
| R-17i-A | 0 | 0 | 0.00 | 0 | |
| R-17i-B | very low | 0.02 | 0.04 | 0.004 | −8 |
| R-17i-C | | 0.04 | 0.08 | 0.008 | −22 |
| R-17i-D | | 0.06 | 0.12 | 0.012 | 7 |
| R-17i-E | | 0.08 | 0.16 | 0.016 | 31 |
| R-17i-F | | 0.1 | 0.20 | 0.02 | 29 |
| R-17i-G | | 0.12 | 0.24 | 0.024 | 99 |
| R-17i-H | | 0.14 | 0.28 | 0.028 | 99 |
| R-17i-I | | 0.2 | 0.40 | 0.04 | 99 |
| R-17i-J | very high | 0.3 | 0.60 | 0.06 | 98 |

DMI = Dry Matter Incubated.
Table 8 formulations were tested 38 days after preparation.

The results demonstrate high anti methanogenic performance (98-99%) for Formulations R8-11, R8-9i and R8-10i, even at very low dosages, and high performance (98-99%) for Formulation R8-17i at medium to high dosages (0.12-0.3 mg bromoform). The formulations of the present invention appear to perform just as well, if not even better, in the presence of the cow rumen microbiome as they do in the presence of the sheep rumen microbiome.

To investigate the performance of the formulations of the invention in the longer term, experiments were conducted using open circuit continuous rumen simulation technique (Rusitec) experiment to find the lowest effective dose that shows a persistent effect on CH₄ in an open fermentation system, and to rule out any potential impacts on rumen microbial activity longer term.

The Rusitec system involves conducting fermentation on a large scale in fermenters filled with rumen fluid and substrate. The Rusitec simulation technique is a well-established in vitro method to simulate and to investigate rumen microbial processes, avoiding animal's variability in a standardized environment. The Rusitec simulation technique provides a more thorough investigation of fermentation characteristics than the batch culture experiments because it is a balanced open fermentation with inputs of nutrient and buffer solutions and outputs of gases, VFA and other fermentation end-products; it is a continuous fermentation over 21 days that allows the examination of treatment effect over time and the adjustment of microbial communities. A major advantage of the Rusitec simulation technique is to give quantitative data on degradability and fermentation for predicting in vivo observations. It is an ‘open system’, meaning that each day fresh substrate is added, and undigested residue removed; there is a continuous flow of ‘saliva’ (buffer); and end-products are regularly/continuously removed from fermenter and measured. This system is closer in replicating in vivo processes, and allows testing the effects over period of time, including persistency of desired effect and/or any negative effects of the treatments on fermentation that may build up over time.

The Rusitec experiments were conducted in accordance with the previously published protocols adapted for testing bioactive feed additives in Garcia et al. 2019, (Garcia, F, Vercoe, PE, Martinez, MJ, Durmic, Z, Brunetti, MA, Moreno, MV, Colombatto, D, Lucini, E, Ferrer, J M (2019) “Essential oils from Lippia turbinata and Tagetes minuta persistently reduce in vitro ruminal methane production in a continuous-culture system.” Animal Production Science 59, 709-720) the contents of which document are hereby incorporated herein in their entirety. The experiment was designed to run for 21 days, with the first 7 days for adaptation of the system using oaten chaff ground to 4 mm as substrate (pre-treatment period; D0-D7), then introducing treatments and running for another 14 days (treatment period, D8-D22). Three treatments were run in triplicate (three fermenters each), and each substrate bag was fermented for 48 h before being replaced with a fresh feed bag. The treatments (1 mL) were added directly onto the top of the fresh feed bag, when placed in the feed container.

Throughout the experiment, pH, total gas and CH₄ concentrations were measured daily in the gas phase, while VFA and NH₃ concentrations from the liquid phase were analysed before introduction of treatments (D7), in the middle (D14) and at the end (D20) of the treatment period. The data were analysed using one-way ANOVA with treatments as factors and true CH₄ concentration, total gas production, total volume of CH₄, mL of CH₄ per gram of dry matter incubated, and pH, VFA and NH₃ concentrations as variables. A covariate was added to the model as an additional predictor. The covariate variables were the same as the treatment variables but for oaten chaff substrate only during the pre-treatment phase (D5 to D7). A Tukey-Kramer HSD pairwise comparison was used when means differed (P<0.05).

The Rusitec simulation was performed for formulations R8-11 and R8-10, and the results are presented in Table 9.

TABLE 9
Rusitec Results

| | | | | | |
|---|---|---|---|---|---|
| | | Bromoform | Final | | |
| | | dose | conc | % | |
| Formulation/ | | (mg) | (g/kg | Bioactive/ | % CH4 |
| Treatment | Dose | per vial | DMI) | DMI | reduction |

After 7 days treatment (D14)

| | | | | | |
|---|---|---|---|---|---|
| Control | none | 0 | | | 0 |
| R8-11-B | very low | 0.6 | 0.04 | 0.004 | 100 |
| R8-11-C | low | 1.2 | 0.08 | 0.008 | 100 |
| R8-11-D | medium | 1.8 | 0.12 | 0.012 | 100 |
| R8-10-C1 | low-daily | 0.9 | 0.06 | 0.006 | 100 |
| R8-10-C1 | low-48 hrs | 0.9 | 0.06 | 0.006 | 75 |

After 14 days treatment (D21)

| | | | | | |
|---|---|---|---|---|---|
| Control | none | 0 | | | 0 |
| R8-11-B | very low | 0.6 | 0.04 | 0.004 | 100 |
| R8-11-C | low | 1.2 | 0.08 | 0.008 | 100 |
| R8-11-D | medium | 1.8 | 0.12 | 0.012 | 100 |
| R8-10-C1 | low-daily | 0.9 | 0.06 | 0.006 | 100 |
| R8-10-C1 | low-48 hrs | 0.9 | 0.06 | 0.006 | 78 |

DMI = Dry Matter Incubated.
Table 9 formulations were tested 60 days after preparation.

In the pre-treatment period, there was no significant difference in total daily gas or CH₄ concentration between groups of fermenters assigned to treatment groups. Mean gas production in this period was 648 mL/24 h, CH₄ concentrations 4.0 mL/100 mL gas and CH₄ production 0.8 mL/g DMi). These levels were retained in the Control throughout the experiment.

The addition of all R8 formulation treatments did not affect overall gas production, but they all significantly (P<0.05) reduced CH₄ concentrations and production compared to Control. The reduction in CH₄ was immediate and after the first day of the addition where CH₄ dropped by an average of 33% across all three R8 treatments. The effect was then cumulative over the first five days of dosing (D8 to D12), declining to 0 mL/100 mL. gas (100% reduction) in all R8-11 treatments by D12. These levels of inhibition persisted for the remainder of the experiment resulting in overall reduction of CH₄ concentrations of 73%-76% in first week and 100% in the second week.

The R8-10 slow-release solid formulation introduced in both fermenters reduced CH₄ immediately, and by 3-4 days, CH₄ was no longer detected in the fermenter with daily supply of the treatment. In the other fermenter where treatment started five days later and was only added every other day, there was a 75% reduction of CH₄ production. The CH₄ inhibition persisted until the conclusion on D22. There was no apparent reduction in total gas production in these.

The pH values were similar between treatments and remained within physiological values (6.5-7.0) at all times throughout the experiment. The VFA profiles were only taken at two timepoints rather than daily due to the high cost of processing and provide a snapshot of the overall trends in VFA concentrations. All treatments produced significantly lower VFA concentrations in week one and two. Interestingly, the control showed a 33% increase of VFA concentrations over time whereas the VFA levels of the treatments remained stable over time, albeit at reduced levels compared to control. The VFA reductions in the treatments compared to control ranged from 26% in the first week in R8-11C, to 45% in the same treatment in the second week compared to the control that also had a 33% VFA increase in week two. After one week of treatment, the reductions of VFA were mainly in acetic and propionic acid, while branch-chained VFA (BCFA) were mostly reduced in the second week. This caused a significant increase in the Acetate:Propionate (A:P) ratios in all R8-11 treatments and in both weeks. The R8-10 formulation, on the other hand, presented dissimilar trends, i.e. the VFA or A:P were not affected with its addition. Propionate and overall VFA were promoted in some instances in this treatment.

The present invention provides potent inhibitors of methanogenesis. Further, as halocarbons are very volatile, it is necessary to stabilize them and deliver them in a carrier formulation that will prevent vaporization and hence loss of activity. The present invention has achieved stabilization, as the effect on CH₄ is evident and sustained over time with the formulations described herein. In all formulations presented, a very potent antimethanogenic effect is delivered (i.e. over 80-90% reduction in CH₄ concentrations), that reaches even total abolishment of CH₄ (100%) over time in the Rusitec simulation. This finding has several implications. Firstly, that it is possible to use a pure, synthetic compound rather than whole seaweed or its crude extract to control enteric CH₄. Secondly, it addresses issues with cost and sustainability of supply, as well as variability that may occur due to varied bromoform levels in different seaweed, along with the prospect of avoiding the environmental impact of seaweed agriculture and harvesting. Finally, it allows a better control over the dose of halocarbon compound to be administered to the ruminant.

### General

Each document, reference, patent application or patent cited in this text is expressly incorporated herein in their entirety by reference, which means that it should be read and considered by the reader as part of this text. That the document, reference, patent application or patent cited in this text is not repeated in this text is merely for reasons of conciseness.

It should be appreciated that throughout this specification, any reference to any prior publication, including prior patent publications and non-patent publications, is not an acknowledgment or admission that any of the material contained within the prior publication referred to was part of the common general knowledge as at the priority date of the application.

Any manufacturer's instructions, descriptions, product specifications, and product sheets for any products mentioned herein or in any document incorporated by reference herein, are hereby incorporated herein by reference, and may be employed in the practice of the invention.

The invention described herein may include one or more range of values (eg. size, displacement and field strength etc). A range of values will be understood to include all values within the range, including the values defining the range, and values adjacent to the range which lead to the same or substantially the same outcome as the values immediately adjacent to that value which defines the boundary to the range.

The present invention is not to be limited in scope by any of the specific embodiments described herein. These embodiments are intended for the purpose of exemplification only. Functionally equivalent products, formulations and methods are clearly within the scope of the invention as described herein.

Those skilled in the art will appreciate that the invention described herein is susceptible to variations and modifications other than those specifically described. The invention includes all such variation and modifications. The invention also includes all of the steps, features, formulations and compounds referred to or indicated in the specification, individually or collectively and any and all combinations or any two or more of the steps or features.

1.-20. (canceled)

21. An edible composition for reducing or inhibiting methanogenesis in a ruminant, the edible composition comprising (a) a stabilized halocarbon and (b) either an emulsion, a micelle, or a liposome, wherein:
(i) either the emulsion, the micelle, or the liposome encapsulates the stabilized halocarbon;
(ii) the edible composition does not comprise red marine macroalgae or a halocarbon producing recombinant yeast;
(iii) the halocarbon compound is not an extract of red marine macroalgae, an extract of a halocarbon producing recombinant yeast, chloroform, carbon tetrachloride, methylene chloride, hexachloroethane, bromochloromethane, bromochloroethane, bromochloropropane, halothane, 2-bromoethane sulphonate, 2-bromo-2-chloroethane sulphonate; and
(iv) the edible composition has a substantially stable concentration of bromoform.

22. The edible composition of claim 21, wherein the stabilized halocarbon is bromoform.

23. The edible composition of claim 21 further comprising a stabilizing agent, wherein the stabilizing agent reduces volatility of the stabilized halocarbon compound in the edible composition as compared to the volatility of an isolated halocarbon.

24. The edible composition of claim 23, wherein the reduced volatility of stabilized halocarbon is measured by comparing the % loss of the stabilized halocarbon compound from the edible composition over a period of time with the % loss of isolated halocarbon from a sample of the isolated halocarbon over the same period of time, and wherein both the edible composition and the isolated halocarbon are stored under identical conditions.

25. The edible composition of claim 23, wherein the stabilizing agent is one or more of: proteins, globular proteins, peptides, oligopeptides, amino acids, carbohydrates, saccharides, disaccharides, oligosaccharides, polysaccharides, xanthan gum, guar gum, celluloses, starches, lipids, fatty acids, waxes, oils, sterols, fat-soluble vitamins, monoglycerides, diglycerides, triglycerides, phospholipids, glycerolipids, sphingolipids, saccharolipids, polyketides, sterol lipids, prenol lipids, emulsifying agents, glycerol, sorbitol, lecithins, glycerophospholipids, phosphatidylcholines, phosphatidylethanolamines, phosphatidylinositols, phosphatidylserines, phosphatidic acids, surfactants, food acids, diacetyl tartaric acid esters of mono- and diglycerides, detergents, encapsulating agents, alkyl celluloses, ethyl celluloses, polyvinyl alcohols, gelatins, carageenans, hydrogels, alginates, alginate salts, edible polymers, biocompatible polymers, fresh milk, or whole milk powder.

26. The edible composition of claim 21, wherein the edible composition is a powder.

27. The edible composition of claim 21, wherein the edible composition is a solid pellet, a pill, or a tablet.

28. The edible composition of claim 27, wherein the edible composition comprises the stabilized halocarbon in rumen of the ruminant and is a slow-release solid composition.

29. The edible composition of claim 28, wherein the slow-release solid composition does not completely dissolve in the rumen of the ruminant for at least 21 days.

30. The edible composition of claim 21, wherein the edible composition does not comprise methylcellulose, polyethlyenemaelic anhydride copolymer, cyclodextrin, or a cyclic oligosaccharide.

31. An edible composition for reducing or inhibiting methanogenesis in a ruminant, the edible composition comprising (a) a stabilized halocarbon and (b) either an emulsion, a micelle, or a liposome, wherein:
(i) either the emulsion, the micelle, or the liposome encapsulates the stabilized halocarbon;
(ii) the edible composition does not comprise red marine macroalgae or a halocarbon producing recombinant yeast; and
(iii) the halocarbon compound is not an extract of red marine macroalgae, an extract of a halocarbon producing recombinant yeast, chloroform, carbon tetrachloride, methylene chloride, hexachloroethane, bromochloromethane, bromochloroethane, bromochloropropane, halothane, 2-bromoethane sulphonate, 2-bromo-2-chloroethane sulphonate.

32. The edible composition of claim 21, wherein the edible composition maintains at least 80% of the concentration of the stabilized halocarbon over a period of up to 7 days.

33. The edible composition of claim 21, wherein the edible composition maintains at least 90% of the concentration of stabilized halocarbon over a period of up to 7 days.

34. A method for reducing or inhibiting methanogenesis in a ruminant, the method comprising administering the edible composition of claim 21 to a ruminant.

35. The method of claim 34, wherein the stabilized halocarbon is bromoform.

36. A method of manufacturing an edible composition for use in reducing or inhibiting methanogenesis in a ruminant, the method comprising:
(a) combining a halocarbon and stabilizing agent; and
(b) encapsulating the combined halocarbon and stabilizing agent by forming an emulsion, a micelle, or a liposome;
wherein the edible composition does not comprise red marine macroalgae or a halocarbon producing recombinant yeast; and
wherein the halocarbon compound is not an extract of red marine macroalgae, an extract of a halocarbon producing recombinant yeast, chloroform, carbon tetrachloride, methylene chloride, hexachloroethane, bromochloromethane, bromochloroethane, bromochloropropane, halothane, 2-bromoethane sulphonate, 2-bromo-2-chloroethane sulphonate.

37. The method of claim 36, wherein the encapsulation is achieved by: pan coating, centrigual extrusion, vibrational nozzle techniques, spray-drying, ionotropic gelation, coacervation-phase separation, interfacial polycondensation, interfacial cross-linking, or matrix polymerization.

38. The method of claim 36, further comprising spray drying the encapsulated combined halocarbon and stabilizing agent.

39. The method of claim 36, further comprising forming a dry powder, a tablet, a pill, or a pellet.

40. The method of claim 36, wherein the halocarbon is bromoform.
